(19) United States
(12) Patent Application Publication
(10) Pub. No.: US 2007/0197389 A1
(43) Pub. Date: Aug. 23, 2007

(54) Azolopyrimidine compounds and use thereof for combating parasitic fungi

(76) Inventors: Anja Schwogler, Mannheim (DE); Markus Gewehr, Kastellaun (DE); Bernd Muller, Frankenthal (DE); Thomas Grote, Wachenheim (DE); Wassilios Grammenos, Ludwigshafen (DE); Jordi Tormo i Blasco, Laudenbach (DE); Joachim Rheinheimer, Ludwigshafen (DE); Carsten Blettner, Mannheim (DE); Peter Schafer, Ottersheim (DE); Frank Schieweck, Hessheim (DE); Oliver Wagner, Neustadt (DE); Reinhard Stierl, Freinsheim (DE); Ulrich Schofl, Bruhl (DE); Siegfried Strathmann, Limburgerhof (DE); Maria Scherer, Godramstein (DE)

Correspondence Address: BIRCH STEWART KOLASCH & BIRCH, PO BOX 747, FALLS CHURCH, VA, 22040-0747, US

(21) Appl. No.: 10/589,496

(22) PCT Filed: Feb. 24, 2005
(86) PCT No.: PCT/EP05/01965

§ 371 Date: Aug. 15, 2006

(30) Foreign Application Priority Data
Feb. 25, 2004 (DE) 10 2004 009 178.1

(51) Int. Cl. A01N 43/713; C07D 487/02

(52) U.S. Cl. 504/191; 544/263

(57) ABSTRACT

The invention relates to azolopyrimidine compounds of general formula (I), wherein A represents N or C—R⁶; X and Y, independent of one another, represent a chemical compound or oxygen, sulphur or a group N—R⁷; the variables R¹, R², R³, R⁴, R⁵, R⁶ and R⁷ have the meanings cited in the claims and the description. The invention also relates to tautomers of compounds of formula (I) and to the agriculturally compatible salts of compounds (I) and of the tautomers thereof. The invention further relates to the use of azolopyrimidine compounds of general formula (I), to the tautomers thereof and to the agriculturally compatible salts thereof which are used to combat phytopathogenic fungi, and to a method for combating phytopathogenic fungi and means for combating fungi, said means containing at least one compound of general formula (I), a tautomer of formula (I) and/or an agriculturally compatible salt thereof or the tautomer thereof and at least one liquid or solid carrier medium.

The present invention relates to novel azolopyrimidine compounds and to their use for controlling harmful fungi, and to crop protection compositions comprising such compounds as active ingredients.

EP-A 71792, U.S. Pat. No. 5,994,360, EP-A 550113, DE-A 10223917, WO 02/48151 and WO 03/080615 describe fungicidally active pyrazolo[1,5-a]pyrimidines and triazolo[1,5a]pyrimidines carrying an optionally substituted phenyl group in the 6-position of the azolopyrimidine ring and NH₂ or a primary or secondary amino group in the 7-position. Similar triazolopyrimidines having, instead of the optionally substituted phenyl ring, an optionally substituted and/or unsaturated aliphatic or cycloaliphatic radical in the 6-position and carrying NH₂ or a primary or secondary amino group in the 7-position are known from WO 03/009687.

Some of the azolopyrimidines known from the prior art are, with respect to their fungicidal action, unsatisfactory, or they have unwanted properties, such as poor crop plant compatibility.

Accordingly, it is an object of the present invention to provide novel compounds having improved fungicidal activity and/or better crop plant compatibility.

Surprisingly, this object is achieved by azolopyrimidine compounds of the formula I 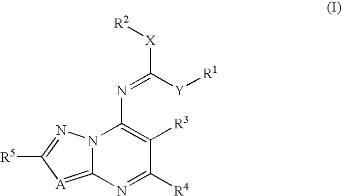
in which
A is N or C—R⁶;
X, Y independently of one another are a chemical bond or oxygen, sulfur or a group N—R⁷;
R¹, R² independently of one another are C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl, naphthyl-C₁-C₄-alkyl, 5- or 6-membered saturated, partially unsaturated or aromatic heterocyclyl or heterocyclyl-C₁-C₄-alkyl which may in each case have 1, 2 or 3 hetero atoms selected from the group consisting of N, O and S as ring members, where some or all of the radicals mentioned as R¹, R² may be halogenated or may have 1, 2, 3 or 4 radicals R⁸, where
   Y—R¹ and X—R² together with the carbon atom, to which they are attached, may also form a 5-, 6- or 7-membered saturated or unsaturated carbo- or heterocycle, where the latter may have 1, 2, 3 or 4 heteroatoms selected from the group consisting of O, S and N as ring members, where the carbo- and the heterocycle may be partially or fully halogenated or have 1, 2, 3 or 4 of the radicals R⁷ and/or R³; where
   Y—R¹ and X—R² independently of one another may also be hydrogen, CN, NO₂ or halogen and where one of the radicals Y—R¹ and X—R² may also be OH, SH or NH₂;
R³ is C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl, a 5- or 6-membered saturated, partially unsaturated or aromatic heterocycle which may have 1, 2 or 3 heteroatoms selected from the group consisting of N, O and S as ring members,
   where the radicals mentioned as R³ may be partially or fully halogenated or may have 1, 2, 3 or 4 radicals R⁹;
R⁴ is halogen, cyano, C₁-C₆-alkyl, C₁-C₆-haloalkyl, C₂-C₆-alkenyl, C₂-C₆-alkynyl, C₃-C₈-cycloalkyl, C₅-C₈-cycloalkenyl, OR¹⁰, SR¹⁰, NR¹¹R¹², CH₂NR¹¹R¹² or C(W)R¹³;
R⁵, R⁶ independently of one another are hydrogen, CN, NO₂, NH₂, CH₂NH₂, halogen, C(W)R¹³, C(═N—OR¹⁵)R¹⁴, NHC(W)R⁶¹, C₁-C₆-haloalkyl, C₁-C₄-alkyl or C₂-C₄-alkenyl;
R⁷ is hydrogen, C₁-C₆-alkyl, C₁-C₆-alkoxy, C₁-C₆-haloalkyl, C₁-C₆-haloalkoxy, C₂-C₆-alkenyl, C₂-C₆-alkenyloxy, CN or C(W)R¹⁷;
R⁸ is selected from the group consisting of halogen, cyano, nitro, OH, SH, NR¹⁸R¹⁹, C₁-C₆-alkyl, C₃-C₈-cycloalkyl, C₁-C₆-alkoxy, hydroxy-C₁-C₆-alkyl, hydroxy-C₁-C₆-alkoxy, C₁-C₆-alkoxy-C₁-C₆-alkyl, C₁-C₆-alkoxy-C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, C(W)R¹³, C(═N—OR¹⁵)R¹⁴, NHC(W)R¹⁶, tris-C₁-C₆-alkylsilyl and phenyl which for its part may have 1, 2 or 3 radicals selected from the group consisting of cyano, nitro, halogen, OH, C₁-C₆-alkyl, C₁-C₆-alkoxy, C₁-C₆-haloalkyl, C₁-C₆-haloalkoxy and C₁-C₆-alkylthio;
R⁹ is halogen, cyano, NH₂, NO₂, C₁-C₆-alkyl, C₃-C₆-cycloalkyl, C₁-C₆-alkoxy, C₁-C₆-haloalkyl, C₁-C₆-haloalkoxy, C₂-C₆-alkenyl, C₂-C₆-alkenyloxy, C(W)R¹³, C(═N—OR¹⁵)R¹⁴ or NHC(W)R¹⁶;
R¹⁰ is hydrogen, C₁-C₆-alkyl, C₁-C₆-haloalkyl, C₂-C₆-alkenyl or C(W)R¹³;
R¹¹, R¹² independently of one another are hydrogen, C₁-C₆-alkyl, C₂-C₆-alkenyl, C₄-C₆-alkadienyl, C₂-C₆-alkynyl, C₃-C₈-cycloalkyl, C₅-C₈-cycloalkenyl, where the radicals mentioned as R¹¹, R¹² may be partially or fully halogenated or have 1, 2, 3 or 4 radicals R⁸, where R¹¹ may also be a group C(W)R¹³ and where
R¹¹, R¹² together with the nitrogen atom, to which they are attached, may also form a 5-, 6- or 7-membered saturated or unsaturated heterocycle which may additionally have 1, 2 or 3 further heteroatoms selected from the group consisting of O, S and N as ring members, where the heterocycle may be partially or fully halogenated and/or may have 1, 2, 3 or 4 of the radicals R⁸;
R¹³ is hydrogen, OH, C₁-C₆-alkyl, C₁-C₆-alkoxy, C₁-C₆-haloalkyl, C₁-C₆-haloalkoxy, C₂-C₆-alkenyl or NR¹⁸R¹⁹;
R¹⁴, R¹⁵ independently of one another are hydrogen or C₁-C₆-alkyl;
R¹⁶, R¹⁷ independently of one another are hydrogen, C₁-C₆-alkyl, C₁-C₆-alkoxy, NH₂, C₁-C₆-alkylamino or di-C₁-C₆-alkylamino;
R¹⁸, R¹⁹ independently of one another have the meanings mentioned for R¹¹ and R¹²; and
W is oxygen or sulfur;
   by the tautomers of the compounds I and by the agriculturally acceptable salts of the compounds I and their tautomers.

The present invention thus provides the azolopyrimidine compounds of the formula I and their agriculturally acceptable salts. The invention also provides their tautomers and the agriculturally acceptable salts of these tautomers.

Tautomers of azolopyrimidine compounds of the formula I are in particular the compounds of the formula II below 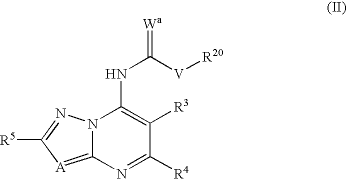
in which A, R³, R⁴ and R⁵ have the meanings given above for formula I,
V is a chemical bond or is oxygen, sulfur or a group N—R⁷;
W^{a} is O, S or a group N—R²¹;
R²⁰ has one of the meanings given in formula I for R¹ or R²;
R²¹ has one of the meanings given in formula I for R¹ or R², where R²¹ may also be hydrogen; and
   if W^{a} is N—R²¹, V—R²⁰ and N—R²¹ together with the carbon atom, to which they are attached, may form a 5-, 6- or 7-membered unsaturated heterocycle, where the latter may have 1, 2, 3 or 4 heteroatoms selected from the group consisting of O, S and N as ring members, may be partially or fully halogenated or have 1, 2, 3 or 4 of the radicals R⁸ mentioned above. These are tautomers of those compounds of the formula I in which one of the radicals Y—R¹ or X—R² is OH, SH, NH₂ or NHR¹ or NHR² (i.e. R⁷ is hydrogen).

Tautomers of compounds of the formula I also include compounds of the formula II′. 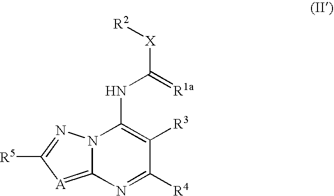
in which A, X, R², R³, R⁴ and R⁵ have the meanings given above and R^{1a} corresponds to the radical R¹ minus one hydrogen atom at the point of attachment. These are tautomers of compounds of the formula I in which Y is a single bond and R¹ has at least one enolizable hydrogen atom. In the tautomers of the formula II′, R^{1a} and X—R² together with the carbon atom, to which they are attached, may also form a 5-, 6- or 7-membered unsaturated carbo- or heterocycle, where the latter may have 1, 2, 3 or 4 heteroatoms selected from the group consisting of O, S and N as ring members, where the carbo- and the heterocycle may be partially or fully halogenated or have 1, 2, 3 or 4 of the radicals R⁷ and/or R⁸ as substitutents.

The present invention furthermore provides the use of the azolopyrimidine compounds of the formula I, their tautomers and their agriculturally acceptable salts for controlling phytopathogenic fungi (=harmful fungi) and a method for controlling phytopathogenic harmful fungi which comprises treating the fungi or the materials, plants, the soil or seeds to be protected against fungal attack with an effective amount of a compound of the formula I, a tautomer of I and/or with an agriculturally acceptable salt of I or its tautomer.

The present invention furthermore provides compositions for controlling harmful fungi, which compositions comprise at least one compound of the formula I, a tautomer of I and/or an agriculturally acceptable salt thereof or of its tautomer and at least one liquid or solid carrier.

Depending on the substitution pattern, the compounds of the formula I and their tautomers may have one or more centers of chirality, in which case they are present as mixtures of enantiomers or diastereomers. The invention provides both the pure enantiomers or diastereomers and their mixtures.

Suitable agriculturally useful salts are especially the salts of those cations or the acid addition salts of those acids whose cations and anions, respectively, have no adverse effect on the fungicidal action of the compounds I or their tautomers. Thus, suitable cations are in particular the ions of the alkali metals, preferably sodium and potassium, of the alkaline earth metals, preferably calcium, magnesium and barium, and of the transition metals, preferably manganese, copper, zinc and iron, and the ammonium ion which, if desired, may carry one to four C₁-C₄-alkyl substitutents and/or one phenyl or benzyl substitutent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen-sulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

In the definitions of the variables given in the above formulae, collective terms are used which are generally representative of the substitutents in question. The term Cₙ-Cₘ indicates the number of carbon atoms possible in each case in the substitutent or substitutent moiety in question:

halogen: fluorine, chlorine, bromine and iodine;

alkyl and the alkyl moieties in alkoxy, alkylthio, alkoxyalkyl, alkoxyalkoxy, alkylamino and dialkylamino: saturated straight-chain or branched hydrocarbon radicals having 1 to 4, up to 6, up to 8 or up to 10 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 4, up to 6, up to 8 or up to 10 carbon atoms (as mentioned above), where some or all of the hydrogen atoms in these groups may be replaced by halogen atoms as mentioned above, for example C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;

alkenyl: monounsaturated straight-chain or branched hydrocarbon radicals having 2 to 4, up to 6, up to 8 or up to 10 carbon atoms and a double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkadienyl: doubly unsaturated straight-chain or branched hydrocarbon radicals having 4 to 10 carbon atoms and two double bonds in any position, for example 1,3-butadienyl, 1-methyl-1,3-butadienyl, 2-methyl-1,3-butadienyl, penta-1,3-dien-1-yl, hexa-1,4-dien-1-yl, hexa-1,4-dien-3-yl, hexa-1,4-dien-6-yl, hexa-1,5-dien-1-yl, hexa-1,5-dien-3-yl, hexa-1,5-dien-4-yl, hepta-1,4-dien-1-yl, hepta-1,4-dien-3-yl, hepta-1,4-dien-6-yl, hepta-1,4-dien-7-yl, hepta-1,5-dien-1-yl, hepta-1,5-dien-3-yl, hepta-1,5-dien-4-yl, hepta-1,5-dien-7-yl, hepta-1,6-dien-1-yl, hepta-1,6-dien-3-yl, hepta-1,6-dien-4-yl, hepta-1,6-dien-5-yl, hepta-1,6-dien-2-yl, octa-1,4-dien-1-yl, octa-1,4-dien-2-yl, octa-1,4-dien-3-yl, octa-1,4-dien-6-yl, octa-1,4-dien-7-yl, octa-1,5-dien-1-yl, octa-1,5-dien-3-yl, octa-1,5-dien-4-yl, octa-1,5-dien-7-yl, octa-1,6-dien-1-yl, octa-1,6-dien-3-yl, octa-1,6-dien-4-yl, octa-1,6-dien-5-yl, octa-1,6-dien-2-yl, deca-1,4-dienyl, deca-1,5-dienyl, deca-1,6-dienyl, deca-1,7-dienyl, deca-1,8-dienyl, deca-2,5-dienyl, deca-2,6-dienyl, deca-2,7-dienyl, deca-2,8-dienyl and the like;

alkynyl: straight-chain or branched hydrocarbon groups having 2 to 4, 2 to 6, 2 to 8 or 2 to 10 carbon atoms and a triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: monocyclic saturated hydrocarbon groups having 3 to 8, preferably up to 6 carbon ring members, such as cyclopropyl, cyclobutyl, cyclopentyl and cyclohexyl;

cycloalkenyl: monocyclic monounsaturated hydrocarbon groups having 5 to 8, preferably up to 6 carbon ring members, such as cyclopenten-1-yl, cyclopenten-3-yl, cyclohexen-1-yl, cyclohexen-3-yl and cyclohexen-4-yl;

bicycloalkyl: a bicyclic hydrocarbon radical having 5 to 10 carbon atoms, such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl and bicyclo[4.4.0]decyl.

alkylamino: an alkyl group attached via an NH group, such as methylamino, ethylamino, n-propylamino, isopropylamino, n-butylamino and the like;

dialkylamino: a radical of the formula N(alkyl)₂, in which alkyl is one of the alkyl radicals mentioned above having generally 1 to 6 and in particular 1 to 4 carbon atoms, for example dimethylamino, diethylamino, methylethylamino, N-methyl-N-propylamino and the like;

C₁-C₄-alkoxy: an alkyl group having 1 to 4 carbon atoms which is attached via oxygen, for example methoxy, ethoxy, n-propoxy, 1-methylethoxy, butoxy, 1-methylpropoxy, 2-methylpropoxy or 1,1-dimethylethoxy;

C₁-C₈-alkoxy: C₁-C₄-alkoxy as mentioned above, and also, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy;

C₁-C₄-haloalkoxy: a C₁-C₄-alkoxy radical as mentioned above which is partially or fully substituted by fluorine, chlorine, bromine and/or iodine, preferably by fluorine, i.e., for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy;

C₁-C₆-haloalkoxy: C₁-C₄-haloalkoxy as mentioned above, and also, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy;

alkenyloxy: alkenyl as mentioned above which is attached via an oxygen atom, for example C₂-C₆-alkenyloxy, such as vinyloxy, 1-propenyloxy, 2-propenyloxy, 1-methylethenyloxy, 1-butenyloxy, 2-butenyloxy, 3-butenyloxy, 1-methyl-1-propenyloxy, 2-methyl-1-propenyloxy, 1-methyl-2-propenyloxy, 2-methyl-2-propenyloxy, 1-pentenyloxy, 2-pentenyloxy, 3-pentenyloxy, 4-pentenyloxy, 1-methyl-1-butenyloxy, 2-methyl-1-butenyloxy, 3-methyl-1-butenyloxy, 1-methyl-2-butenyloxy, 2-methyl-2-butenyloxy, 3-methyl-2-butenyloxy, 1-methyl-3-butenyloxy, 2-methyl-3-butenyloxy, 3-methyl-3-butenyl, 1,1-dimethyl-2-propenyloxy, 1,2-dimethyl-1-propenyloxy, 1,2-dimethyl-2-propenyloxy, 1-ethyl-1-propenyloxy, 1-ethyl-2-propenyloxy, 1-hexenyloxy, 2-hexenyloxy, 3-hexenyloxy, 4-hexenyloxy, 5-hexenyloxy, 1-methyl-1-pentenyloxy, 2-methyl-1-pentenyloxy, 3-methyl-1-pentenyloxy, 4-methyl-1-pentenyloxy, 1-methyl-2-pentenyloxy, 2-methyl-2-pentenyloxy, 3-methyl-2-pentenyloxy, 4-methyl-2-pentenyloxy, 1-methyl-3-pentenyloxy, 2-methyl-3-pentenyloxy, 3-methyl-3-pentenyloxy, 4-methyl-3-pentenyloxy, 1-methyl-4-pentenyloxy, 2-methyl-4-pentenyloxy, 3-methyl-4-pentenyloxy, 4-methyl-4-pentenyloxy, 1,1-dimethyl-2-butenyloxy, 1,1-dimethyl-3-butenyloxy, 1,2-dimethyl-1-butenyloxy, 1,2-dimethyl-2-butenyloxy, 1,2-dimethyl-3-butenyloxy, 1,3-dimethyl-1-butenyloxy, 1,3-dimethyl-2-butenyloxy, 1,3-dimethyl-3-butenyloxy, 2,2-dimethyl-3-butenyloxy, 2,3-dimethyl-1-butenyloxy, 2,3-dimethyl-2-butenyloxy, 2,3-dimethyl-3-butenyloxy, 3,3-dimethyl-1-butenyloxy, 3,3-dimethyl-2-butenyloxy, 1-ethyl-1-butenyloxy, 1-ethyl-2-butenyloxy, 1-ethyl-3-butenyloxy, 2-ethyl-1-butenyloxy, 2-ethyl-2-butenyloxy, 2-ethyl-3-butenyloxy, 1,1,2-trimethyl-2-propenyloxy, 1-ethyl-1-methyl-2-propenyloxy, 1-ethyl-2-methyl-1-propenyloxy and 1-ethyl-2-methyl-2-propenyloxy;

alkynyloxy: alkynyl as mentioned above which is attached via an oxygen atom, for example C₃-C₆-alkynyloxy, such as 2-propynyloxy, 2-butynyloxy, 3-butynyloxy, 1-methyl-2-propynyloxy, 2-pentynyloxy, 3-pentynyloxy, 4-pentynyloxy, 1-methyl-2-butynyloxy, 1-methyl-3-butynyloxy, 2-methyl-3-butynyloxy, 1-ethyl-2-propynyloxy, 2-hexynyloxy, 3-hexynyloxy, 4-hexynyloxy, 5-hexynyloxy, 1-methyl-2-pentynyloxy, 1-methyl-3-pentynyloxy and the like;

a five- to seven-membered saturated, partially unsaturated or aromatic heterocycle or heterocyclyl which contains one, two or three heteroatoms from the group consisting of O, N and S:

a saturated, partially unsaturated (for example monounsaturated) or aromatic heterocyclic radical having 5, 6 or 7 ring atoms, 1, 2 or 3 of which are selected from the group consisting of nitrogen, oxygen and sulfur and the remaining ring atoms are carbon, for example:
5- or 6-membered saturated or monounsaturated heterocyclyl which contains one to two nitrogen atoms and/or one oxygen or sulfur atom or one or two oxygen and/or sulfur atoms as ring members, for example 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 1-piperidinyl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered aromatic heterocyclyl (=heteroaryl or hetaryl) which, in addition to carbon atoms, contains one, two or three nitrogen atoms or one or two nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;
6-membered heterocyclyl (=heteroaryl or hetaryl) which, in addition to carbon atoms, contains one or two or one, two or three nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,2,4-triazin-3-yl; 1,2,4-triazin-5-yl, 1,2,4-triazin-6-yl and 1,3,5-triazin-2-yl.

A first embodiment of the invention relates to compounds of the formula I in which A is N. Hereinbelow, such compounds are also referred to as compounds I-A. A second embodiment of the invention relates to compounds of the formula I in which A is C—R⁶. Hereinbelow, such compounds are also referred to as compounds I-B.

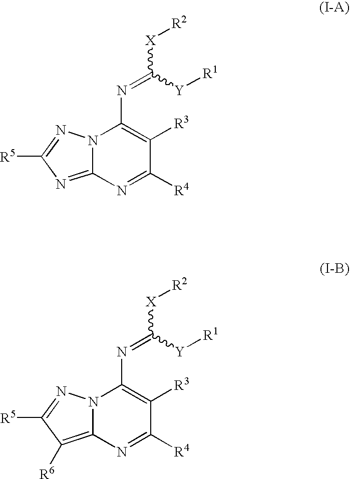

With a view to the fungicidal action of the compounds according to the invention, preference is given to those compounds of the formula I in which A, R¹, R², R³, R⁴ and R⁵ have in particular the meanings indicated below:

R¹ and R² independently of one another are C₁-C₁₀-alkyl, C₁-C₁₀-haloalkyl, C₃-C₁₀-alkenyl, C₃-C₁₀-haloalkenyl, C₃-C₈-cycloalkyl, C₅-C₈-cycloalkenyl, C₃-C₈-cycloalkyl-C₁-C₁₀-alkyl, C₃-C₈-cycloalkyl-C₂-C₁₀-alkenyl, phenyl or benzyl, where the 6 lastmentioned radicals may also carry 1, 2, 3 or 4 substitutents selected from the group consisting of halogen, C₁-C₄-alkyl, C₁-C₄-haloalkyl and C₁-C₄-alkoxy, or a group X—R² or Y—R¹ is hydrogen or halogen, especially chlorine, and the remaining radical R² or R¹ has the meanings indicated here as being preferred.

Hereinbelow, preferred groups R¹ and R² are illustrated in more detail. What is stated below for R¹ applies correspondingly also to R². R¹ is preferably C₁-C₄-alkyl, C₂-C₆-alkenyl or C₁-C₉-haloalkyl. If R¹ is an alkyl, alkenyl or alkynyl group, this can be branched at the α carbon atom. In these cases, the group R¹ corresponds to a group C: 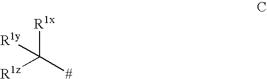
in which # is the bond to the carbon atom of the imino group or to Y and
R^{1x} is C₁-C₃-alkyl or C₁-C₃-haloalkyl;
R^{1y} is hydrogen, C₁-C₃-alkyl or C₁-C₃-haloalkyl;
R^{1z} is C₁-C₈-alkyl, C₂-C₈-alkenyl or C₂-C₈-alkynyl, where R^{1z} may be unsubstituted or partially or fully halogenated and/or may carry one to three groups R⁸.

Preference is likewise given to compounds I in which R¹ is a 5- or 6-membered saturated or aromatic heterocycle which contains one or two heteroatoms from the group consisting of N, O and S and which may be substituted by one or two alkyl or haloalkyl groups.

Preference is given to compounds I in which R¹ is a group B: 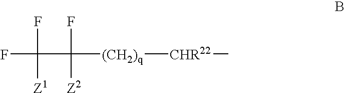
in which
Z¹ is hydrogen, fluorine or C₁-C₆-fluoroalkyl,
Z² is hydrogen or fluorine, or
Z¹ and Z² together form a double bond;
q is 0 or 1; and
R²² is hydrogen or methyl.

Moreover, preference is given to compounds I in which R¹ is C₃-C₆-cycloalkyl which may be substituted by C₁-C₄-alkyl.

If X—R² and Y—R¹ and the carbon atom, to which they are attached, form an optionally substituted carbo- or heterocycle, this cycle is preferably selected from among 5-, 6- or 7-membered saturated or monounsaturated cycles which optionally include one heteroatom as ring member. In this case, for example, X—R² and Y—R¹ together are —(CH₂)₂CH═CHCH₂—, —(CH₂)₂C(CH₃)═CHCH₂—, —(CH₂)₂CH(CH₃)(CH₂)—, —(CH₂)₂CHF(CH₂)—, —(CH₂)₃CHFCH₂—, —(CH₂)₂CH(CF₃)(CH₂)₂—, —(CH₂)₂—O—(CH₂)₂—, —(CH₂)₂S(CH₂)₂—, —(CH₂)₅—, —(CH₂)₆—, —(CH₂)₄—, —CH₂CH═CHCH₂—, —CH(CH₃)(CH₂)₃—, —CH(CH₃)(CH₂)—, —CH₂CH(CH₃)(CH₂)₂— or —CH₂CH(CH₃)(CH₂)₃—.

Among the compounds of the formula I, preference is furthermore given to those in which R³ is a phenyl ring which has 1, 2, 3 or 4, in particular 1, 2 or 3, of the radicals R⁹ indicated above or is pentafluorophenyl. Preferably, at least one of the radicals R⁹ is located in the ortho-position to the point of attachment. In this case, R⁹ is selected in particular from among the following radicals: halogen, especially fluorine or chlorine, CN, C₁-C₄-alkyl, especially methyl or ethyl, C₁-haloalkyl, especially trifluoromethyl, C₁-C₄-alkoxy, especially methoxy or —C(═O)—R¹³ in which R¹³ has the meanings indicated above and is in particular hydrogen, hydroxyl, C₁-C₄-alkoxy, C₁-C₄-haloalkoxy, C₁-C₂-alkylamino or di-C₁-C₂-alkylamino. Among these, preference is given to those compounds of the formula I in which R³ is a group of the formula 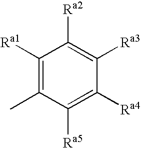
in which
R^{a1} is fluorine, chlorine, methyl or CF₃;
R^{a2} is hydrogen, chlorine or fluorine;
R^{a3} is hydrogen, CN, NO₂, fluorine, chlorine, C₁-C₄-alkyl, especially methyl, C₁-C₄-alkoxy, especially methoxy or a group C(W)R^{13a} in which W is oxygen or sulfur and R^{13a} is C₁-C₄-alkoxy, NH₂, C₁-C₄-alkylamino or di-C₁-C₄-alkylamino, the group C(W)R^{13a} being especially C(O)OCH₃, CONH₂, C(S)OCH₃;
R^{a4} is hydrogen, chlorine or fluorine;
R^{a5} is hydrogen, fluorine, chlorine or C₁-C₄-alkyl.

Among the compounds of the formula I, preference is furthermore given to those compounds in which R³ is an optionally substituted hydrocarbon radical having 3 to 8 carbon atoms and in particular optionally substituted C₃-C₆-cycloalkyl, C₃-C₆-cycloalkylmethyl, C₃-C₈-alkyl, C₁-C₈-haloalkyl or benzyl and, for example, propyl, isopropyl, isobutyl, 1-methylbutyl, tert-butyl, n-octyl, cyclopropyl, cyclopropylmethyl, cyclopentyl, cyclohexyl, 2,2,2-trifluoroethyl, benzyl or 2-, 3- or 4-chlorophenylmethyl.

Among the compounds of the formula I, preference is furthermore given to those compounds in which R³ is a 5- or 6-membered heteroaromatic radical which has 1, 2 or 3 heteroatoms selected from the group consisting of N, O and S as ring members and which may have 1, 2, 3 or 4 radicals R⁹.

Examples of heterocyclic radicals on R³ are 1-, 2- or 3-pyrazolyl, 2- or 3-thienyl, for example 4-thiazolyl, isothiazolyl, for example 4-isothiazolyl, oxazolyl, for example 4-oxazolyl, isoxazolyl, for example 4-isoxazolyl, pyrrolyl, for example 2-pyrrolyl, imidazolyl, for example 1-imidazolyl, pyridyl, for example 2-, 3-, or 4-pyridyl, pyrazinyl, for example 2-pyrazinyl, pyridazine, for example 3-pyridazinyl, pyrimidinyl, for example 2-, 4- or 5-pyrimidinyl and 1,3,5-triazin-2-yl, where the radicals mentioned above may be unsubstituted or, depending on the number of carbon atoms in the ring, may have 1, 2, 3 or 4 radicals R⁹. Preferred radicals R⁹ are halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl.

Preferred heteroaromatic radicals include the radicals het-1 to het-21 shown below: 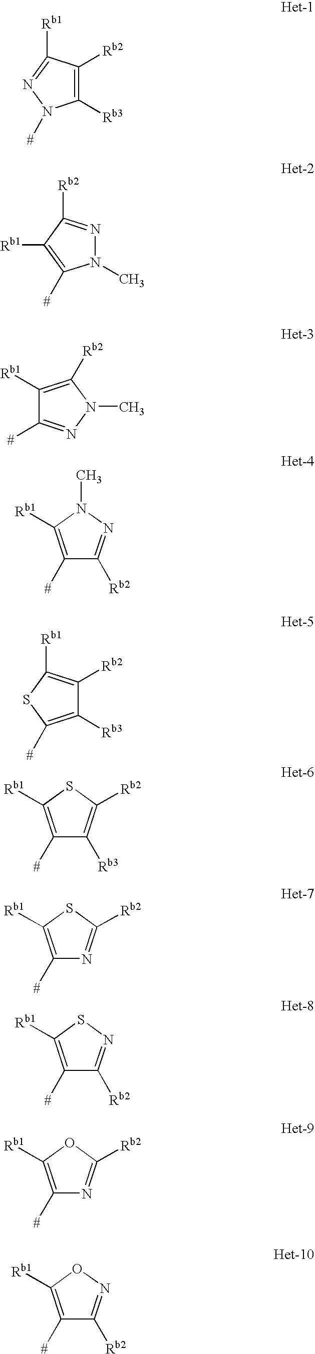 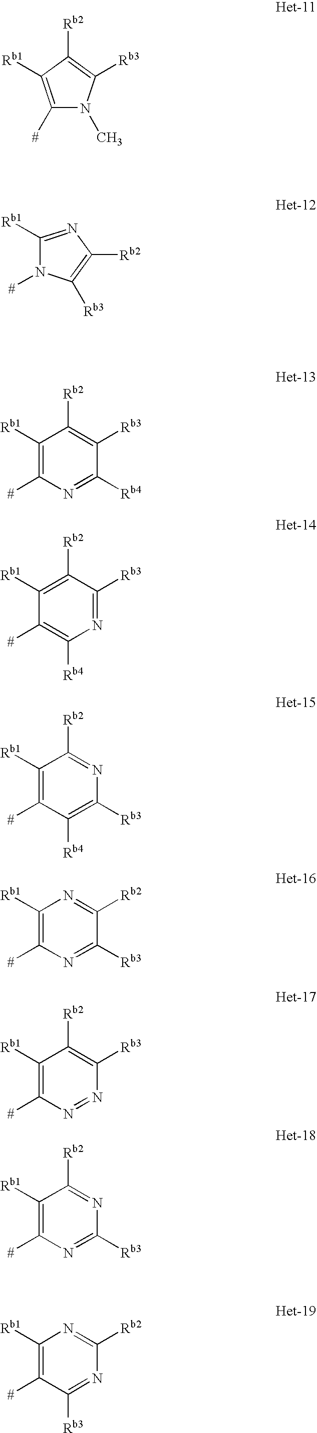 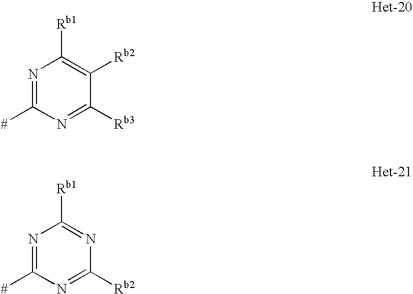
in which
# denotes the point of attachment; and
R^{b1}, R^{b2}, R^{b3} and R^{b4} independently of one another are hydrogen or have the meanings mentioned for R⁹.

Preferably, the radicals R^{b1}, R^{b2}, R^{b3} and R^{b4} independently of one another are selected from the group consisting of hydrogen, halogen, nitro, cyano, C₁-C₄-alkyl, C₁-C₄-haloalkyl, especially C₁-C₂-fluoroalkyl, C₁-C₄-alkoxy and C₁-C₄-alkoxycarbonyl. In a particularly preferred embodiment, R^{b1}, R^{b2}, R^{b3} and R^{b4} independently of one another are selected from the group consisting of hydrogen, nitro, cyano, fluorine, chlorine, bromine, methyl, ethyl, isopropyl, trifluoromethyl, fluoromethyl, methoxy and methoxycarbonyl.

Examples of het-1 include 3,5-dimethylpyrazol-1-yl, 3,5-diisopropylpyrazol-1-yl, 3-methyl-5-isopropylpyrazol-1-yl, 3-isopropyl-5-methylpyrazol-1-yl, 3-ethyl-5-methylpyrazol-1-yl, 3,4,5-trimethylpyrazol-1-yl, 3-trifluoromethylpyrazol-1-yl, 3-trifluoromethyl-5-methoxypyrazol-1-yl, 3-trifluoromethyl-5-methylpyrazol-1-yl, 3-methyl-5-methoxypyrazol-1-yl, 3,5-dimethyl-4-chloropyrazol-1-yl and 3,5-ditrifluoromethylpyrazol-1-yl.

Examples of het-2 include 1,3-dimethylpyrazol-5-yl and 1-methyl-3-trifluoromethylpyrazol-1-yl.

Examples of het-3 include 1,5-dimethylpyrazol-3-yl and 1-methyl-5-methoxypyrazol-3-yl.

Examples of het-4 include 1,3-dimethylpyrazol-4-yl, 1,5-dimethylpyrazol-4-yl, 1,3,5-trimethylpyrazol-4-yl, 1-methyl-3-trifluoromethylpyrazol-4-yl and 1-methyl-5-trifluoromethylpyrazol-4-yl.

Examples of het-5 include 2-thienyl, 5-methylthiophen-2-yl, 5-chlorothiophen-2-yl, 3,5-dichlorothiophen-2-yl, 3,4,5-trichlorothiophen-2-yl and 5-bromothiophen-2-yl.

Examples of het-6 include 3-thienyl, 2-methylthiophen-3-yl, 2,5-dichlorothiophen-3-yl, 2,4,5-trichlorothiophen-3-yl and 2,5-dibromothiophen-3-yl.

Examples of het-7 include thiazol-4-yl, 2-methylthiazol-4-yl, 2-methyl-5-chlorothiazol-4-yl and 2,5-dichlorothiazol-4-yl.

Examples of het-8 include 3-methylisothiazol-4-yl and 3-methyl-5-chloroisothiazol-4-yl.

Examples of het-9 include oxazol-4-yl, 2-methyloxazol-4-yl and 2,5-dimethyloxazol-4-yl.

Examples of het-10 include isoxazol-4-yl, 3,5-dimethylisoxazol-4-yl and 3-chloroisoxazol-4-yl.

Examples of het-11 include 1-methylpyrrol-2-yl, 1,4-dimethylpyrrol-2-yl, 1-methyl-5-chloropyrrol-2-yl and 1-methyl-3,5-dichloropyrrol-2-yl.

Examples of het-12 include 4,5-dichloroimidazol-1-yl and 4,5-dimethylimidazol-1-yl.

Examples of het-13 include 2-pyridyl, 3-fluoropyridin-2-yl, 3,5-difluoropyridin-2-yl, 3,5-dichloropyridin-2-yl, 3-fluoro-5-trifluoromethylpyridin-2-yl, 3-trifluoromethylpyridin-2-yl, 5-nitropyridin-2-yl, 5-cyanopyridin-2-yl, 5-methoxycarbonylpyridin-2-yl, 5-trifluoromethylpyridin-2-yl, 5-methylpyridin-2-yl, 4-methylpyridin-2-yl, 3-methylpyridin-2-yl, 3-ethylpyridin-2-yl and 6-methylpyridin-2-yl.

An example of het-14 is 3-pyridyl.

An example of het-15 is 4-pyridyl.

An example of het-16 is pyrazin-2-yl.

Examples of het-17 include pyridazin-3-yl, 6-chloropyridazin-3-yl, 6-methoxypyridazin-3-yl.

Examples of het-18 include 5-chloropyrimidin-4-yl, 5-fluoropyrimidin-4-yl, 5-fluoro-6-chloropyrimidin-4-yl, 2-methyl-6-trifluoromethyl-pyrimidin-4-yl, 2,5-dimethyl-6-trifluoromethylpyrimidin-4-yl, 5-methyl-6-trifluoromethylpyrimidin-4-yl, 6-trifluoromethylpyrimidin-4-yl, 2-methyl-5-fluoropyrimidin-4-yl, 2-methyl-5-chloropyrimidin-4-yl, 5-chloro-6-methylpyrimidin-4-yl, 5-chloro-6-ethylpyrimidin-4-yl, 5-chloro-6-isopropylpyrimidin-4-yl, 5-bromo-6-methylpyrimidin-4-yl, 5-fluoro-6-methylpyrimidin-4-yl, 5-fluoro-6-fluoromethylpyrimidin-4-yl, 2,6-dimethyl-5-chloropyrimidin-4-yl, 5,6-dimethylpyrimidin-4-yl, 2,5-dimethylpyrimidin-4-yl, 2,5,6-trimethylpyrimidin-4-yl and 5-methyl-6-methoxypyrimidin-4-yl.

Examples of het-19 include 4-methylpyrimidin-5-yl, 4,6-dimethylpyrimidin-5-yl, 2,4,6-trimethylpyrimidin-5-yl and 4-trifluoromethyl-6-methylpyrimidin-5-yl.

Examples of het-20 include 4,6-dimethylpyrimidin-2-yl, 4,5,6-trimethylpyrimidin-2-yl, 4,6-ditrifluoromethylpyrimidin-2-yl and 4,6-dimethyl-5-chloropyrimidin-2-yl.

An example of het-21 is 1,3,5-triazin-2-yl.

Furthermore, it has been found to be advantageous for R⁴ in formula I to be halogen, CN, C₁-C₄-alkoxy, especially methoxy, or C₁-C₄-alkyl, especially methyl. Among these, preference is given in particular to compounds of the formula I in which R⁴ is halogen. Preference is also given to compounds of the formula I in which R⁴ is methyl or methoxy.

Among the compounds of the formula I, preference is furthermore given to those compounds in which R⁵ is hydrogen, halogen, especially chlorine or fluorine, or C₁-C₄-alkyl, especially methyl. In a particularly preferred embodiment, R⁵ is hydrogen.

In the compounds of the formula I-B, R⁶ is preferably hydrogen, halogen, especially chlorine or fluorine, a group C(W)R^{13b} in which W is oxygen or sulfur and R^{13b} is C₁-C₄-alkoxy, NH₂, C₁-C₄-alkylamino or di-C₁-C₄-alkylamino, especially C(O)OCH₃, CONH₂, C(S)OCH₃, or C₁-C₄-alkyl, especially methyl. If R⁵ is different from hydrogen, R⁶ is in particular hydrogen. With particular preference, R⁵ and R⁶ in formula I-B are hydrogen.

In a preferred embodiment of the compounds according to the invention, at least one of the variables X or Y in formula I is a chemical bond. Among these, preference is given to those compounds in which one of the groups Y—R¹ or X—R² is hydrogen or C₁-C₈-alkyl and especially C₁-C₄-alkyl. The other of these groups Y—R¹ or X—R² has the meanings indicated above. In this case, R¹ and R² have in particular one of the meanings indicated as being preferred.

In a particularly preferred embodiment of the compounds I, both variables X and Y are a chemical bond. In this case, R¹ and R² independently of one another have the meanings indicated above and are in particular selected from the group consisting of hydrogen, C₁-C₁₀-alkyl, C₁-C₁₀-haloalkyl, C₃-C₁₀-alkenyl, C₃-C₁₀-haloalkenyl, C₃-C₈-cycloalkyl, C₅₃-C₈-cycloalkenyl, C₃-C₈-cycloalkyl-C₁-C₁₀-alkyl, C₃-C₈-cycloalkyl-C₂-C₁₀-alkenyl, phenyl and benzyl, where the 6 lastmentioned radicals may also carry 1, 2, 3 or 4 substitutents selected from the group consisting of halogen, C₁-C₄-alkyl, C₁-C₄-haloalkyl and C₁-C₄-alkoxy, where one of the radicals R¹ or R² may also be halogen and especially chlorine. Among these, particular preference is given to those compounds in which one of the radicals R¹ or R² is a group of the formula C or B as defined above.

Among the compounds I in which X and Y are each a chemical bond, preference is given to those compounds in which one of the variables R¹ or R² is hydrogen or C₁-C₄-alkyl and the other variable has one of the meanings indicated above, in particular a meaning mentioned as being preferred.

Among the compounds I in which X and Y are each a chemical bond, preference is furthermore given to those compounds in which one of the variables R¹ or R² is halogen, especially is chlorine, and the other variable has one of the meanings indicated above, in particular a meaning mentioned as being preferred.

R⁷ is in particular hydrogen or C₁-C₄-alkyl. Compounds where R⁷=hydrogen can in particular also be present in the form of tautomers of the formula II in which W^{a} is a group N—R²¹.

In a further preferred embodiment of the compounds according to the invention, one of the variables X or Y in formula I is a group NR⁷. Among these, preference is given to those compounds I in which Y is N—R⁷, where R⁷ has the meanings mentioned above and in particular a meaning mentioned as being preferred. In the group —(NR⁷)—R¹, R¹ is then C₁-C₁₀-alkyl, C₂-C₁₀-alkenyl, C₄-C₁₀-alkadienyl, C₂-C₁₀alkynyl, C₃-C₈-cycloalkyl, C₅-C₈-cycloalkenyl, C₅-C₁₀-bicycloalkyl, phenyl, phenyl-C₁-C₄-alkyl, naphthyl, naphthyl-C₁-C₄-alkyl, where the radicals mentioned as R¹ may be partially or fully halogenated and/or may have 1, 2, 3 or 4 radicals R⁸. Very particular preference is given to compounds of the formula I in which the group (NR⁷)R¹ is C₁-C₆-alkylamino or di-C₁-C₆-alkylamino, especially C₁-C₄-alkylamino or di-C₁-C₄-alkylamino.

Preference is likewise given to compounds I in which in the group —(NR⁷)—R¹ the substitutents R¹ and R⁷ together with the nitrogen atom to which they are attached are a 5- or 6-membered saturated, partially unsaturated or aromatic N-heterocycle which may have one or two further heteroatoms selected from the group consisting of O, S and N as ring member and/or may have 1, 2, 3 or 4 radicals R⁸, in which R⁸ has one or of the meanings mentioned above and in particular one of the meanings mentioned as being preferred.

Among these, particular preference is given to those compounds I in which the group —(NR⁷)—R¹ is 5- or 6-membered saturated heterocyclyl which is attached via nitrogen, which optionally has a further heteroatom selected from the group consisting of N, O and S as ring atom and which optionally carries 1, 2, 3 or 4 substitutents R⁸ selected from the group consisting of halogen and C₁-C₄-alkyl. In a particularly preferred embodiment, the group —(NR⁷)—R¹ is piperidin-1-yl, 4-methyl-1-piperidinyl, 1-pyrrolidinyl, 2,5-dihydropyrrol-1-yl, 4-morpholinyl or 4-thiomorpholinyl.

Preference is likewise given to compounds I in which X is a chemical bond, R² is hydrogen or C₁-C₄-alkyl and the group —(NR⁷)—R¹ has one of the meanings mentioned above and in particular one of the meanings mentioned as being preferred.

R⁸ is in particular halogen, especially fluorine, C₁-C₄-alkoxy or C₁-C₄-alkyl.

In the groups OR¹⁰, SR¹⁰, NR¹¹R¹², C(W)R¹³, C(═N—OR¹⁵)R¹⁴, NHC(W)R¹⁶, C(W)R¹⁷ and NR¹⁸R¹⁹, the variables have in particular the meanings indicated below:

R¹⁰ is in particular H, C₁-C₄-alkyl, C(O)H or C₁-C₄-alkylcarbonyl. OR¹⁰ is in particular OH, C₁-C₄-alkoxy, O—C(O)H or C₁-C₄-alkylcarbonyloxy. OR¹⁰ is in particular SH or S—C₁-C₄-alkyl.

R¹¹ and R¹² are in particular H, C₁-C₄-alkyl, C₁-C₄-alkylcarbonyl or C₁-C₄-alkyl(thiocarbonyl). NR¹¹R¹² is in particular NH₂, NHCH₃, NHC₂H₅, N(CH₃)₂, N(C₂H₅)CH₃, NHC(O)CH₃ or NHC(O)H.

R¹³ is in particular H, C₁-C₄-alkyl, OH, NH₂, NHCH₃, NHC₂H₅, N(CH₃)₂, N(C₂H₅)CH₃ or C₁-C₄-alkoxy.

R¹⁴ is in particular C₁-C₄-alkyl.

R¹⁵ is in particular C₁-C₄-alkyl.

R¹⁶ is in particular hydrogen or C₁-C₄-alkyl.

R¹⁷ is in particular H, C₁-C₄-alkyl or C₁-C₄-alkoxy.

R¹⁸ and R¹⁹ are in particular H, C₁-C₄-alkyl, C₁-C₄-alkylcarbonyl or C₁-C₄-alkyl(thiocarbonyl). NR¹⁸R¹⁹ is in particular NH₂, NHCH₃, NHC₂H₅, N(CH₃)₂, N(C₂H₅)CH₃, NHC(O)CH₃ or NHC(O)H.

Particularly preferred compounds of the formula I are the triazolopyrimidines of the formula I-A in which R³ is 2-fluoro-6-chlorophenyl, R⁴ is chlorine, R⁵ is hydrogen and X, Y, R¹ and R² have the meanings mentioned above and in particular the meanings mentioned as being preferred (compounds I-A1). Examples of these are the compounds I-A1.1 to I-A1.414 in which X—R² and Y—R¹ together have in each case the meanings given in one row of Table A.

Particularly preferred compounds of the formula I are also the triazolopyrimidines of the formula I-A in which R³ is 2,6-difluorophenyl, R⁴ is chlorine, R⁵ is hydrogen and X, Y, R¹ and R² have the meanings mentioned above and in particular the meanings mentioned as being preferred (compounds I-A2). Examples of these are the compounds I-A2.1 to I-A2.414 in which X—R² and Y—R¹ together have in each case the meanings given in one row of Table A.

Particularly preferred compounds of the formula I are also the triazolopyrimidines of the formula I-A in which R³ is 2,6-dichlorophenyl, R⁴ is chlorine, R⁵ is hydrogen and X, Y, R¹ and R² have the meanings mentioned above and in particular the meanings mentioned as being preferred (compounds I-A3). Examples of these are the compounds I-A3.1 to I-A3.414 in which X—R² and Y—R¹ together have in each case the meanings given in one row of Table A.

Particularly preferred compounds of the formula I are also the triazolopyrimidines of the formula I-A in which R³ is 2-fluoro-6-methylphenyl, R⁴ is chlorine, R⁵ is hydrogen and X, Y, R¹ and R² have the meanings mentioned above and in particular the meanings mentioned as being preferred (compounds I-A4). Examples of these are the compounds I-A4.1 to I-A4.414 in which X—R² and Y—R¹ together have in each case the meanings given in one row of Table A.

Particularly preferred compounds of the formula I are also the triazolopyrimidines of the formula I-A in which R³ is 2,4,6-trifluorophenyl, R⁴ is chlorine, R⁵ is hydrogen and X, Y, R¹ and R² have the meanings mentioned above and in particular the meanings mentioned as being preferred (compounds I-A5). Examples of these are the compounds I-A5.1 to I-A5.414 in which X—R² and Y—R¹ together have in each case the meanings given in one row of Table A.

Particularly preferred compounds of the formula I are also the triazolopyrimidines of the formula I-A in which R³ is 2,6-difluoro-4-methoxyphenyl, R⁴ is chlorine, R⁵ is hydrogen and X, Y, R¹ and R² have the meanings mentioned above and in particular the meanings mentioned as being preferred (compounds I-A6). Examples of these are the compounds I-A6.1 to I-A6.414 in which X—R² and Y—R¹ together have in each case the meanings given in one row of Table A.

Particularly preferred compounds of the formula I are also the triazolopyrimidines of the formula I-A in which R³ is 2-chloro-6-methylphenyl, R⁴ is chlorine, R⁵ is hydrogen and X, Y, R¹ and R² have the meanings mentioned above and in particular the meanings mentioned as being preferred (compounds I-A7). Examples of these are the compounds I-A7.1 to I-A7.414 in which X—R² and Y—R¹ together have in each case the meanings given in one row of Table A.

Particularly preferred compounds of the formula I are also the triazolopyrimidines of the formula I-A in which R³ is pentafluorophenyl, R⁴ is chlorine, R⁵ is hydrogen and X, Y, R¹ and R² have the meanings mentioned above and in particular the meanings mentioned as being preferred (compounds I-A8). Examples of these are the compounds I-A8.1 to I-A8.414 in which X—R² and Y—R¹ together have in each case the meanings given in one row of Table A.

Particularly preferred compounds of the formula I are also the triazolopyrimidines of the formula I-A in which R³ is 2-methyl-4-fluorophenyl, R⁴ is chlorine, R⁵ is hydrogen and X, Y, R¹ and R² have the meanings mentioned above and in particular the meanings mentioned as being preferred (compounds I-A9). Examples of these are the compounds I-A9.1 to I-A9.414 in which X—R² and Y—R¹ together have in each case the meanings given in one row of Table A.

Particularly preferred compounds of the formula I are also the triazolopyrimidines of the formula I-A in which R³ is 2-trifluoromethylphenyl, R⁴ is chlorine, R⁵ is hydrogen and X, Y, R¹ and R² have the meanings mentioned above and in particular the meanings mentioned as being preferred (compounds I-A10). Examples of these are the compounds I-A10.1 to I-A10.414 in which X—R² and Y—R¹ together have in each case the meanings given in one row of Table A.

Particularly preferred compounds of the formula I are also the triazolopyrimidines of the formula I-A in which R³ is 2-methoxy-6-fluorophenyl, R⁴ is chlorine, R⁵ is hydrogen and X, Y, R¹ and R² have the meanings mentioned above and in particular the meanings mentioned as being preferred (compounds I-A11). Examples of these are the compounds I-A11.1 to I-A11.414 in which X—R² and Y—R¹ together have in each case the meanings given in one row of Table A.

Particularly preferred compounds of the formula I are also the triazolopyrimidines of the formula I-A in which R³ is 2-chlorophenyl, R⁴ is chlorine, R⁵ is hydrogen and X, Y, R¹ and R² have the meanings mentioned above and in particular the meanings mentioned as being preferred (compounds I-A12). Examples of these are the compounds I-A12.1 to I-A12.414 in which X—R² and Y—R¹ together have in each case the meanings given in one row of Table A.

Particularly preferred compounds of the formula I are also the triazolopyrimidines of the formula I-A in which R³ is 2-fluorophenyl, R⁴ is chlorine, R⁵ is hydrogen and X, Y, R¹ and R² have the meanings mentioned above and in particular the meanings mentioned as being preferred (compounds I-A13). Examples of these are the compounds I-A13.1 to I-A13.414 in which X—R² and Y—R¹ together have in each case the meanings given in one row of Table A.

Particularly preferred compounds of the formula I are also the triazolopyrimidines of the formula I-A in which R³ is 2,4-difluorophenyl, R⁴ is chlorine, R⁵ is hydrogen and X, Y, R¹ and R² have the meanings mentioned above and in particular the meanings mentioned as being preferred (compounds I-A14). Examples of these are the compounds I-A14.1 to I-A14.414 in which X—R² and Y—R¹ together have in each case the meanings given in one row of Table A.

Particularly preferred compounds of the formula I are also the triazolopyrimidines of the formula I-A in which R³ is 2-fluoro-4-chlorophenyl, R⁴ is chlorine, R⁵ is hydrogen and X, Y, R¹ and R² have the meanings mentioned above and in particular the meanings mentioned as being preferred (compounds I-A15). Examples of these are the compounds I-A15.1 to I-A15.414 in which X—R² and Y—R¹ together have in each case the meanings given in one row of Table A.

Particularly preferred compounds of the formula I are also the triazolopyrimidines of the formula I-A in which R³ is 4-fluoro-6-chlorophenyl, R⁴ is chlorine, R⁵ is hydrogen and X, Y, R¹ and R² have the meanings mentioned above and in particular the meanings mentioned as being preferred (compounds I-A16). Examples of these are the compounds I-A16.1 to I-A16.414 in which X—R² and Y—R¹ together have in each case the meanings given in one row of Table A.

Particularly preferred compounds of the formula I are also the triazolopyrimidines of the formula I-A in which R³ is 2,3-difluorophenyl, R⁴ is chlorine, R⁵ is hydrogen and X, Y, R¹ and R² have the meanings mentioned above and in particular the meanings mentioned as being preferred (compounds I-A17). Examples of these are the compounds I-A17.1 to I-A17.414 in which X—R² and Y—R¹ together have in each case the meanings given in one row of Table A.

Particularly preferred compounds of the formula I are also the triazolopyrimidines of the formula I-A in which R³ is 2,5-difluorophenyl, R⁴ is chlorine, R⁵ is hydrogen and X, Y, R¹ and R² have the meanings mentioned above and in particular the meanings mentioned as being preferred (compounds I-A18). Examples of these are the compounds I-A18.1 to I-A18.414 in which X—R² and Y—R¹ together have in each case the meanings given in one row of Table A.

Particularly preferred compounds of the formula I are also the triazolopyrimidines of the formula I-A in which R³ is 2,3,4-trifluorophenyl, R⁴ is chlorine, R⁵ is hydrogen and X, Y, R¹ and R² have the meanings mentioned above and in particular the meanings mentioned as being preferred (compounds I-A19). Examples of these are the compounds I-A19.1 to I-A19.414 in which X—R² and Y—R¹ together have in each case the meanings given in one row of Table A.

Particularly preferred compounds of the formula I are also the triazolopyrimidines of the formula I-A in which R³ is 2-methylphenyl, R⁴ is chlorine, R⁵ is hydrogen and X, Y, R¹ and R² have the meanings mentioned above and in particular the meanings mentioned as being preferred (compounds I-A20). Examples of these are the compounds I-A20.1 to I-A20.414 in which X—R² and Y—R¹ together have in each case the meanings given in one row of Table A.

Particularly preferred compounds of the formula I are also the triazolopyrimidines of the formula I-A in which R³ is 2,4-dimethylphenyl, R⁴ is chlorine, R⁵ is hydrogen and X, Y, R¹ and R² have the meanings mentioned above and in particular the meanings mentioned as being preferred (compounds I-A21). Examples of these are the compounds I-A21.1 to I-A21.414 in which X—R² and Y—R¹ together have in each case the meanings given in one row of Table A.

Particularly preferred compounds of the formula I are also the triazolopyrimidines of the formula I-A in which R³ is 2-methyl-4-chlorophenyl, R⁴ is chlorine, R⁵ is hydrogen and X, Y, R¹ and R² have the meanings mentioned above and in particular the meanings mentioned as being preferred (compounds I-A22). Examples of these are the compounds I-A22.1 to I-A22.414 in which X—R² and Y—R¹ together have in each case the meanings given in one row of Table A.

Particularly preferred compounds of the formula I are also the triazolopyrimidines of the formula I-A in which R³ is 2-fluoro-4-methylphenyl, R⁴ is chlorine, R⁵ is hydrogen and X, Y, R¹ and R² have the meanings mentioned above and in particular the meanings mentioned as being preferred (compounds I-A23). Examples of these are the compounds I-A23.1 to I-A23.414 in which X—R² and Y—R¹ together have in each case the meanings given in one row of Table A.

Particularly preferred compounds of the formula I are also the triazolopyrimidines of the formula I-A in which R³ is 2,6-dimethylphenyl, R⁴ is chlorine, R⁵ is hydrogen and X, Y, R¹ and R² have the meanings mentioned above and in particular the meanings mentioned as being preferred (compounds I-A24). Examples of these are the compounds I-A24.1 to I-A24.414 in which X—R² and Y—R¹ together have in each case the meanings given in one row of Table A.

Particularly preferred compounds of the formula I are also the triazolopyrimidines of the formula I-A in which R³ is 2,4,5-trimethylphenyl, R⁴ is chlorine, R⁵ is hydrogen and X, Y, R¹ and R² have the meanings mentioned above and in particular the meanings mentioned as being preferred (compounds I-A25). Examples of these are the compounds I-A25.1 to I-A25.414 in which X—R² and Y—R¹ together have in each case the meanings given in one row of Table A.

Particularly preferred compounds of the formula I are also the triazolopyrimidines of the formula I-A in which R³ is 2,6-difluoro-4-cyanophenyl, R⁴ is chlorine, R⁵ is hydrogen and X, Y, R¹ and R² have the meanings mentioned above and in particular the meanings mentioned as being preferred (compounds I-A26). Examples of these are the compounds I-A26.1 to I-A26.414 in which X—R² and Y—R¹ together have in each case the meanings given in one row of Table A.

Particularly preferred compounds of the formula I are also the triazolopyrimidines of the formula I-A in which R³ is 2,6-difluoro-4-methylphenyl, R⁴ is chlorine, R⁵ is hydrogen and X, Y, R¹ and R² have the meanings mentioned above and in particular the meanings mentioned as being preferred (compounds I-A27). Examples of these are the compounds I-A27.1 to I-A27.414 in which X—R² and Y—R¹ together have in each case the meanings given in one row of Table A.

Particularly preferred compounds of the formula I are also the triazolopyrimidines of the formula I-A in which R³ is 2,6-difluoro-4-methoxycarbonylphenyl, R⁴ is chlorine, R⁵ is hydrogen and X, Y, R¹ and R² have the meanings mentioned above and in particular the meanings mentioned as being preferred (compounds I-A28). Examples of these are the compounds I-A28.1 to I-A28.414 in which X—R² and Y—R¹ together have in each case the meanings given in one row of Table A.

Particularly preferred compounds of the formula I are also the triazolopyrimidines of the formula I-A in which R³ is 2-trifluoromethyl-4-fluorophenyl, R⁴ is chlorine, R⁵ is hydrogen and X, Y, R¹ and R² have the meanings mentioned above and in particular the meanings mentioned as being preferred (compounds I-A29). Examples of these are the compounds I-A29.1 to I-A29.414 in which X—R² and Y—R¹ together have in each case the meanings given in one row of Table A.

Particularly preferred compounds of the formula I are also the triazolopyrimidines of the formula I-A in which R³ is 2-trifluoromethyl-5-fluorophenyl, R⁴ is chlorine, R⁵ is hydrogen and X, Y, R¹ and R² have the meanings mentioned above and in particular the meanings mentioned as being preferred (compounds I-A30). Examples of these are the compounds I-A30.1 to I-A30.414 in which X—R² and Y—R¹ together have in each case the meanings given in one row of Table A.

Particularly preferred compounds of the formula I are also the triazolopyrimidines of the formula I-A in which R³ is 2-trifluoromethyl-5-chlorophenyl, R⁴ is chlorine, R⁵ is hydrogen and X, Y, R¹ and R² have the meanings mentioned above and in particular the meanings mentioned as being preferred (compounds I-A31). Examples of these are the compounds I-A31.1 to I-A31.414 in which X—R² and Y—R¹ together have in each case the meanings given in one row of Table A.

Particularly preferred compounds of the formula I are the triazolopyrimidines of the formula I-A in which R³ is 2-fluoro-6-chlorophenyl, R⁴ is methyl, R⁵ is hydrogen and X, Y, R¹ and R² have the meanings mentioned above and in particular the meanings mentioned as being preferred (compounds I-A32). Examples of these are the compounds I-A32.1 to I-A32.414 in which X—R² and Y—R¹ together have in each case the meanings given in one row of Table A.

Particularly preferred compounds of the formula I are also the triazolopyrimidines of the formula I-A in which R³ is 2,6-difluorophenyl, R⁴ is methyl, R⁵ is hydrogen and X, Y, R¹ and R² have the meanings mentioned above and in particular the meanings mentioned as being preferred (compounds I-A33). Examples of these are the compounds I-A33.1 to I-A33.414 in which X—R² and Y—R¹ together have in each case the meanings given in one row of Table A.

Particularly preferred compounds of the formula I are also the triazolopyrimidines of the formula I-A in which R³ is 2,6-dichlorophenyl, R⁴ is methyl, R⁵ is hydrogen and X, Y, R¹ and R² have the meanings mentioned above and in particular the meanings mentioned as being preferred (compounds I-A34). Examples of these are the compounds I-A34.1 to I-A34.414 in which X—R² and Y—R¹ together have in each case the meanings given in one row of Table A.

Particularly preferred compounds of the formula I are also the triazolopyrimidines of the formula I-A in which R³ is 2-fluoro-6-methylphenyl, R⁴ is methyl, R⁵ is hydrogen and X, Y, R¹ and R² have the meanings mentioned above and in particular the meanings mentioned as being preferred (compounds I-A35). Examples of these are the compounds I-A35.1 to I-A35.414 in which X—R² and Y—R¹ together have in each case the meanings given in one row of Table A.

Particularly preferred compounds of the formula I are also the triazolopyrimidines of the formula I-A in which R³ is 2,4,6-trifluorophenyl, R⁴ is methyl, R⁵ is hydrogen and X, Y, R¹ and R² have the meanings mentioned above and in particular the meanings mentioned as being preferred (compounds I-A36). Examples of these are the compounds I-A36.1 to I-A36.414 in which X—R² and Y—R¹ together have in each case the meanings given in one row of Table A.

Particularly preferred compounds of the formula I are also the triazolopyrimidines of the formula I-A in which R³ is 2,6-difluoro-4-methoxyphenyl, R⁴ is methyl, R⁵ is hydrogen and X, Y, R¹ and R² have the meanings mentioned above and in particular the meanings mentioned as being preferred (compounds I-A37). Examples of these are the compounds I-A37.1 to I-A37.414 in which X—R² and Y—R¹ together have in each case the meanings given in one row of Table A.

Particularly preferred compounds of the formula I are also the triazolopyrimidines of the formula I-A in which R³ is 2-chloro-6-methylphenyl, R⁴ is methyl, R⁵ is hydrogen and X, Y, R¹ and R² have the meanings mentioned above and in particular the meanings mentioned as being preferred (compounds I-A38). Examples of these are the compounds I-A38.1 to I-A38.414 in which X—R² and Y—R¹ together have in each case the meanings given in one row of Table A.

Particularly preferred compounds of the formula I are also the triazolopyrimidines of the formula I-A in which R³ is pentafluorophenyl, R⁴ is methyl, R⁵ is hydrogen and X, Y, R¹ and R² have the meanings mentioned above and in particular the meanings mentioned as being preferred (compounds I-A39). Examples of these are the compounds I-A39.1 to I-A39.414 in which X—R² and Y—R¹ together have in each case the meanings given in one row of Table A.

Particularly preferred compounds of the formula I are also the triazolopyrimidines of the formula I-A in which R³ is 2-methyl-4-fluorophenyl, R⁴ is methyl, R⁵ is hydrogen and X, Y, R¹ and R² have the meanings mentioned above and in particular the meanings mentioned as being preferred (compounds I-A40). Examples of these are the compounds I-A40.1 to I-A40.414 in which X—R² and Y—R¹ together have in each case the meanings given in one row of Table A.

Particularly preferred compounds of the formula I are also the triazolopyrimidines of the formula I-A in which R³ is 2-trifluoromethylphenyl, R⁴ is methyl, R⁵ is hydrogen and X, Y, R¹ and R² have the meanings mentioned above and in particular the meanings mentioned as being preferred (compounds I-A41). Examples of these are the compounds I-A41.1 to I-A41.414 in which X—R² and Y—R¹ together have in each case the meanings given in one row of Table A.

Particularly preferred compounds of the formula I are also the triazolopyrimidines of the formula I-A in which R³ is 2-methoxy-6-fluorophenyl, R⁴ is methyl, R⁵ is hydrogen and X, Y, R¹ and R² have the meanings mentioned above and in particular the meanings mentioned as being preferred (compounds I-A42). Examples of these are the compounds I-A42.1 to I-A42.414 in which X—R² and Y—R¹ together have in each case the meanings given in one row of Table A.

Particularly preferred compounds of the formula I are also the triazolopyrimidines of the formula I-A in which R³ is 2-chlorophenyl, R⁴ is methyl, R⁵ is hydrogen and X, Y, R¹ and R² have the meanings mentioned above and in particular the meanings mentioned as being preferred (compounds I-A43). Examples of these are the compounds I-A43.1 to I-A43.414 in which X—R² and Y—R¹ together have in each case the meanings given in one row of Table A.

Particularly preferred compounds of the formula I are also the triazolopyrimidines of the formula I-A in which R³ is 2-fluorophenyl, R⁴ is methyl, R⁵ is hydrogen and X, Y, R¹ and R² have the meanings mentioned above and in particular the meanings mentioned as being preferred (compounds I-A44). Examples of these are the compounds I-A44.1 to I-A44.414 in which X—R² and Y—R¹ together have in each case the meanings given in one row of Table A.

Particularly preferred compounds of the formula I are also the triazolopyrimidines of the formula I-A in which R³ is 2,4-difluorophenyl, R⁴ is methyl, R⁵ is hydrogen and X, Y, R¹ and R² have the meanings mentioned above and in particular the meanings mentioned as being preferred (compounds I-A45). Examples of these are the compounds I-A45.1 to I-A45.414 in which X—R² and Y—R¹ together have in each case the meanings given in one row of Table A.

Particularly preferred compounds of the formula I are also the triazolopyrimidines of the formula I-A in which R³ is 2-fluoro-4-chlorophenyl, R⁴ is methyl, R⁵ is hydrogen and X, Y, R¹ and R² have the meanings mentioned above and in particular the meanings mentioned as being preferred (compounds I-A46). Examples of these are the compounds I-A46.1 to I-A46.414 in which X—R² and Y—R¹ together have in each case the meanings given in one row of Table A.

Particularly preferred compounds of the formula I are also the triazolopyrimidines of the formula I-A in which R³ is 4-fluoro-6-chlorophenyl, R⁴ is methyl, R⁵ is hydrogen and X, Y, R¹ and R² have the meanings mentioned above and in particular the meanings mentioned as being preferred (compounds I-A47). Examples of these are the compounds I-A47.1 to I-A47.414 in which X—R² and Y—R¹ together have in each case the meanings given in one row of Table A.

Particularly preferred compounds of the formula I are also the triazolopyrimidines of the formula I-A in which R³ is 2,3-difluorophenyl, R⁴ is methyl, R⁵ is hydrogen and X, Y, R¹ and R² have the meanings mentioned above and in particular the meanings mentioned as being preferred (compounds I-A48). Examples of these are the compounds I-A48.1 to I-A48.414 in which X—R² and Y—R¹ together have in each case the meanings given in one row of Table A.

Particularly preferred compounds of the formula I are also the triazolopyrimidines of the formula I-A in which R³ is 2,5-difluorophenyl, R⁴ is methyl, R⁵ is hydrogen and X, Y, R¹ and R² have the meanings mentioned above and in particular the meanings mentioned as being preferred (compounds I-A49). Examples of these are the compounds I-A49.1 to I-A49.414 in which X—R² and Y—R¹ together have in each case the meanings given in one row of Table A.

Particularly preferred compounds of the formula I are also the triazolopyrimidines of the formula I-A in which R³ is 2,3,4-trifluorophenyl, R⁴ is methyl, R⁵ is hydrogen and X, Y, R¹ and R² have the meanings mentioned above and in particular the meanings mentioned as being preferred (compounds I-A50). Examples of these are the compounds I-A50.1 to I-A50.414 in which X—R² and Y—R¹ together have in each case the meanings given in one row of Table A.

Particularly preferred compounds of the formula I are also the triazolopyrimidines of the formula I-A in which R³ is 2-methylphenyl, R⁴ is methyl, R⁵ is hydrogen and X, Y, R¹ and R² have the meanings mentioned above and in particular the meanings mentioned as being preferred (compounds I-A51). Examples of these are the compounds I-A51.1 to I-A51.414 in which X—R² and Y—R¹ together have in each case the meanings given in one row of Table A.

Particularly preferred compounds of the formula I are also the triazolopyrimidines of the formula I-A in which R³ is 2,4-dimethylphenyl, R⁴ is methyl, R⁵ is hydrogen and X, Y, R¹ and R² have the meanings mentioned above and in particular the meanings mentioned as being preferred (compounds I-A51). Examples of these are the compounds I-A51.1 to I-A51.414 in which X—R² and Y—R¹ together have in each case the meanings given in one row of Table A.

Particularly preferred compounds of the formula I are also the triazolopyrimidines of the formula I-A in which R³ is 2-methyl-4-chlorophenyl, R⁴ is methyl, R⁵ is hydrogen and X, Y, R¹ and R² have the meanings mentioned above and in particular the meanings mentioned as being preferred (compounds I-A52). Examples of these are the compounds I-A52.1 to I-A52.414 in which X—R² and Y—R¹ together have in each case the meanings given in one row of Table A.

Particularly preferred compounds of the formula I are also the triazolopyrimidines of the formula I-A in which R³ is 2-fluoro-4-methylphenyl, R⁴ is methyl, R⁵ is hydrogen and X, Y, R¹ and R² have the meanings mentioned above and in particular the meanings mentioned as being preferred (compounds I-A53). Examples of these are the compounds I-A53.1 to I-A53.414 in which X—R² and Y—R¹ together have in each case the meanings given in one row of Table A.

Particularly preferred compounds of the formula I are also the triazolopyrimidines of the formula I-A in which R³ is 2,6-dimethylphenyl, R⁴ is methyl, R⁵ is hydrogen and X, Y, R¹ and R² have the meanings mentioned above and in particular the meanings mentioned as being preferred (compounds I-A54). Examples of these are the compounds I-A54.1 to I-A54.414 in which X—R² and Y—R¹ together have in each case the meanings given in one row of Table A.

Particularly preferred compounds of the formula I are also the triazolopyrimidines of the formula I-A in which R³ is 2,4,5-trimethylphenyl, R⁴ is methyl, R⁵ is hydrogen and X, Y, R¹ and R² have the meanings mentioned above and in particular the meanings mentioned as being preferred (compounds I-A55). Examples of these are the compounds I-A55.1 to I-A55.414 in which X—R² and Y—R¹ together have in each case the meanings given in one row of Table A.

Particularly preferred compounds of the formula I are also the triazolopyrimidines of the formula I-A in which R³ is 2,6-difluoro-4-cyanophenyl, R⁴ is methyl, R⁵ is hydrogen and X, Y, R¹ and R² have the meanings mentioned above and in particular the meanings mentioned as being preferred (compounds I-A56). Examples of these are the compounds I-A56.1 to I-A56.414 in which X—R² and Y—R¹ together have in each case the meanings given in one row of Table A.

Particularly preferred compounds of the formula I are also the triazolopyrimidines of the formula I-A in which R³ is 2,6-difluoro-4-methylphenyl, R⁴ is methyl, R⁵ is hydrogen and X, Y, R¹ and R² have the meanings mentioned above and in particular the meanings mentioned as being preferred (compounds I-A57). Examples of these are the compounds I-A57.1 to I-A57.414 in which X—R² and Y—R¹ together have in each case the meanings given in one row of Table A.

Particularly preferred compounds of the formula I are also the triazolopyrimidines of the formula I-A in which R³ is 2,6-difluoro-4-methoxycarbonylphenyl, R⁴ is methyl, R⁵ is hydrogen and X, Y, R¹ and R² have the meanings mentioned above and in particular the meanings mentioned as being preferred (compounds I-A58). Examples of these are the compounds I-A58.1 to I-A58.414 in which X—R² and Y—R¹ together have in each case the meanings given in one row of Table A.

Particularly preferred compounds of the formula I are also the triazolopyrimidines of the formula I-A in which R³ is 2-trifluoromethyl-4-fluorophenyl, R⁴ is methyl, R⁵ is hydrogen and X, Y, R¹ and R² have the meanings mentioned above and in particular the meanings mentioned as being preferred (compounds I-A59). Examples of these are the compounds I-A59.1 to I-A59.414 in which X—R² and Y—R¹ together have in each case the meanings given in one row of Table A.

Particularly preferred compounds of the formula I are also the triazolopyrimidines of the formula I-A in which R³ is 2-trifluoromethyl-5-fluorophenyl, R⁴ is methyl, R⁵ is hydrogen and X, Y, R¹ and R² have the meanings mentioned above and in particular the meanings mentioned as being preferred (compounds I-A60). Examples of these are the compounds I-A60.1 to I-A60.414 in which X—R² and Y—R¹ together have in each case the meanings given in one row of Table A.

Particularly preferred compounds of the formula I are also the triazolopyrimidines of the formula I-A in which R³ is 2-trifluoromethyl-5-chlorophenyl, R⁴ is methyl, R⁵ is hydrogen and X, Y, R¹ and R² have the meanings mentioned above and in particular the meanings mentioned as being preferred (compounds I-A61). Examples of these are the compounds I-A61.1 to I-A61.414 in which X—R² and Y—R¹ together have in each case the meanings given in one row of Table A.

Particularly preferred compounds of the formula I are the pyrazolopyrimidines of the formula I-B in which R³ is 2-fluoro-6-chlorophenyl, R⁴ is chlorine, R⁵ is hydrogen and X, Y, R¹ and R² have the meanings mentioned above and in particular the meanings mentioned as being preferred (compounds I-B1). Examples of these are the compounds I-B1.1 to I-B1.414 in which X—R² and Y—R¹ together have in each case the meanings given in one row of Table A.

Particularly preferred compounds of the formula I are also the pyrazolopyrimidines of the formula I-B in which R³ is 2,6-difluorophenyl, R⁴ is chlorine, R⁵ is hydrogen and X, Y, R¹ and R² have the meanings mentioned above and in particular the meanings mentioned as being preferred (compounds I-B2). Examples of these are the compounds I-B2.1 to I-B2.414 in which X—R² and Y—R¹ together have in each case the meanings given in one row of Table A.

Particularly preferred compounds of the formula I are also the pyrazolopyrimidines of the formula I-B in which R³ is 2,6-dichlorophenyl, R⁴ is chlorine, R⁵ is hydrogen and X, Y, R¹ and R² have the meanings mentioned above and in particular the meanings mentioned as being preferred (compounds I-B3). Examples of these are the compounds I-B3.1 to I-B3.414 in which X—R² and Y—R¹ together have in each case the meanings given in one row of Table A.

Particularly preferred compounds of the formula I are also the pyrazolopyrimidines of the formula I-B in which R³ is 2-fluoro-6-methylphenyl, R⁴ is chlorine, R⁵ is hydrogen and X, Y, R¹ and R² have the meanings mentioned above and in particular the meanings mentioned as being preferred (compounds I-B4). Examples of these are the compounds I-B4.1 to I-B4.414 in which X—R² and Y—R¹ together have in each case the meanings given in one row of Table A.

Particularly preferred compounds of the formula I are also the pyrazolopyrimidines of the formula I-B in which R³ is 2,4,6-trifluorophenyl, R⁴ is chlorine, R⁵ is hydrogen and X, Y, R¹ and R² have the meanings mentioned above and in particular the meanings mentioned as being preferred (compounds I-B5). Examples of these are the compounds I-B5.1 to I-B5.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methoxyphenyl, R⁴ is chlorine, R⁵ is hydrogen and X, Y, R¹ and R² have the meanings mentioned above and in particular the meanings mentioned as being preferred (compounds I-B6). Examples of these are the compounds I-B6.1 to I-B6.414 in which X—R² and Y—R¹ together have in each case the meanings given in one row of Table A.

Particularly preferred compounds of the formula I are also the pyrazolopyrimidines of the formula I-B in which R³ is 2-chloro-6-methylphenyl, R⁴ is chlorine, R⁵ is hydrogen and X, Y, R¹ and R² have the meanings mentioned above and in particular the meanings mentioned as being preferred (compounds I-B7). Examples of these are the compounds I-B7.1 to I-B7.414 in which X—R² and Y—R¹ together have in each case the meanings given in one row of Table A.

Particularly preferred compounds of the formula I are also the pyrazolopyrimidines of the formula I-B in which R³ is pentafluorophenyl, R⁴ is chlorine, R⁵ is hydrogen and X, Y, R¹ and R² have the meanings mentioned above and in particular the meanings mentioned as being preferred (compounds I-B8). Examples of these are the compounds I-B8.1 to I-B8.414 in which X—R² and Y—R¹ together have in each case the meanings given in one row of Table A.

Particularly preferred compounds of the formula I are also the pyrazolopyrimidines of the formula I-B in which R³ is 2-methyl-4-fluorophenyl, R⁴ is chlorine, R⁵ is hydrogen and X, Y, R¹ and R² have the meanings mentioned above and in particular the meanings mentioned as being preferred (compounds I-B9). Examples of these are the compounds I-B9.1 to I-B9.414 in which X—R² and Y—R¹ together have in each case the meanings given in one row of Table A.

Particularly preferred compounds of the formula I are also the pyrazolopyrimidines of the formula I-B in which R³ is 2-trifluoromethylphenyl, R⁴ is chlorine, R⁵ is hydrogen and X, Y, R¹ and R² have the meanings mentioned above and in particular the meanings mentioned as being preferred (compounds I-B10). Examples of these are the compounds I-B10.1 to I-B10.414 in which X—R² and Y—R¹ together have in each case the meanings given in one row of Table A.

Particularly preferred compounds of the formula I are also the pyrazolopyrimidines of the formula I-B in which R³ is 2-methoxy-6-fluorophenyl, R⁴ is chlorine, R⁵ is hydrogen and X, Y, R¹ and R² have the meanings mentioned above and in particular the meanings mentioned as being preferred (compounds I-B11). Examples of these are the compounds I-B11.1 to I-B11.414 in which X—R² and Y—R¹ together have in each case the meanings given in one row of Table A.

Particularly preferred compounds of the formula I are also the pyrazolopyrimidines of the formula I-B in which R³ is 2-chlorophenyl, R⁴ is chlorine, R⁵ is hydrogen and X, Y, R¹ and R² have the meanings mentioned above and in particular the meanings mentioned as being preferred (compounds I-B12). Examples of these are the compounds I-B12.1 to I-B12.414 in which X—R² and Y—R¹ together have in each case the meanings given in one row of Table A.

Particularly preferred compounds of the formula I are also the pyrazolopyrimidines of the formula I-B in which R³ is 2-fluorophenyl, R⁴ is chlorine, R⁵ is hydrogen and X, Y, R¹ and R² have the meanings mentioned above and in particular the meanings mentioned as being preferred (compounds I-B13). Examples of these are the compounds I-B13.1 to I-B13.414 in which X—R² and Y—R¹ together have in each case the meanings given in one row of Table A.

Particularly preferred compounds of the formula I are also the pyrazolopyrimidines of the formula I-B in which R³ is 2,4-difluorophenyl, R⁴ is chlorine, R⁵ is hydrogen and X, Y, R¹ and R² have the meanings mentioned above and in particular the meanings mentioned as being preferred (compounds I-B14). Examples of these are the compounds I-B14.1 to I-B14.414 in which X—R² and Y—R¹ together have in each case the meanings given in one row of Table A.

Particularly preferred compounds of the formula I are also the pyrazolopyrimidines of the formula I-B in which R³ is 2-fluoro-4-chlorophenyl, R⁴ is chlorine, R⁵ is hydrogen and X, Y, R¹ and R² have the meanings mentioned above and in particular the meanings mentioned as being preferred (compounds I-B15). Examples of these are the compounds I-B15.1 to I-B15.414 in which X—R² and Y—R¹ together have in each case the meanings given in one row of Table A.

Particularly preferred compounds of the formula I are also the pyrazolopyrimidines of the formula I-B in which R³ is 4-fluoro-6-chlorophenyl, R⁴ is chlorine, R⁵ is hydrogen and X, Y, R¹ and R² have the meanings mentioned above and in particular the meanings mentioned as being preferred (compounds I-B16). Examples of these are the compounds I-B16.1 to I-B16.414 in which X—R² and Y—R¹ together have in each case the meanings given in one row of Table A.

Particularly preferred compounds of the formula I are also the pyrazolopyrimidines of the formula I-B in which R³ is 2,3-difluorophenyl, R⁴ is chlorine, R⁵ is hydrogen and X, Y, R¹ and R² have the meanings mentioned above and in particular the meanings mentioned as being preferred (compounds I-B17). Examples of these are the compounds I-B17.1 to I-B17.414 in which X—R² and Y—R¹ together have in each case the meanings given in one row of Table A.

Particularly preferred compounds of the formula I are also the pyrazolopyrimidines of the formula I-B in which R³ is 2,5-difluorophenyl, R⁴ is chlorine, R⁵ is hydrogen and X, Y, R¹ and R² have the meanings mentioned above and in particular the meanings mentioned as being preferred (compounds I-B18). Examples of these are the compounds I-B18.1 to I-B18.414 in which X—R² and Y—R¹ together have in each case the meanings given in one row of Table A.

Particularly preferred compounds of the formula I are also the pyrazolopyrimidines of the formula I-B in which R³ is 2,3,4-trifluorophenyl, R⁴ is chlorine, R⁵ is hydrogen and X, Y, R¹ and R² have the meanings mentioned above and in particular the meanings mentioned as being preferred (compounds I-B19). Examples of these are the compounds I-B19.1 to I-B19.414 in which X—R² and Y—R¹ together have in each case the meanings given in one row of Table A.

Particularly preferred compounds of the formula I are also the pyrazolopyrimidines of the formula I-B in which R³ is 2-methylphenyl, R⁴ is chlorine, R⁵ is hydrogen and X, Y, R¹ and R² have the meanings mentioned above and in particular the meanings mentioned as being preferred (compounds I-B20). Examples of these are the compounds I-B20.1 to I-B20.414 in which X—R² and Y—R¹ together have in each case the meanings given in one row of Table A.

Particularly preferred compounds of the formula I are also the pyrazolopyrimidines of the formula I-B in which R³ is 2,4-dimethylphenyl, R⁴ is chlorine, R⁵ is hydrogen and X, Y, R¹ and R² have the meanings mentioned above and in particular the meanings mentioned as being preferred (compounds I-B21). Examples of these are the compounds I-B21.1 to I-B21.414 in which X—R² and Y—R¹ together have in each case the meanings given in one row of Table A.

Particularly preferred compounds of the formula I are also the pyrazolopyrimidines of the formula I-B in which R³ is 2-methyl-4-chlorophenyl, R⁴ is chlorine, R⁵ is hydrogen and X, Y, R¹ and R² have the meanings mentioned above and in particular the meanings mentioned as being preferred (compounds I-B22). Examples of these are the compounds I-B22.1 to I-B22.414 in which X—R² and Y—R¹ together have in each case the meanings given in one row of Table A.

Particularly preferred compounds of the formula I are also the pyrazolopyrimidines of the formula I-B in which R³ is 2-fluoro-4-methylphenyl, R⁴ is chlorine, R⁵ is hydrogen and X, Y, R¹ and R² have the meanings mentioned above and in particular the meanings mentioned as being preferred (compounds I-B23). Examples of these are the compounds I-B23.1 to I-B23.414 in which X—R² and Y—R¹ together have in each case the meanings given in one row of Table A.

Particularly preferred compounds of the formula I are also the pyrazolopyrimidines of the formula I-B in which R³ is 2,6-dimethylphenyl, R⁴ is chlorine, R⁵ is hydrogen and X, Y, R¹ and R² have the meanings mentioned above and in particular the meanings mentioned as being preferred (compounds I-B24). Examples of these are the compounds I-B24.1 to I-B24.414 in which X—R² and Y—R¹ together have in each case the meanings given in one row of Table A.

Particularly preferred compounds of the formula I are also the pyrazolopyrimidines of the formula I-B in which R³ is 2,4,5-trimethylphenyl, R⁴ is chlorine, R⁵ is hydrogen and X, Y, R¹ and R² have the meanings mentioned above and in particular the meanings mentioned as being preferred (compounds I-B25). Examples of these are the compounds I-B25.1 to I-B25.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-cyanophenyl, R⁴ is chlorine, R⁵ is hydrogen and X, Y, R¹ and R² have the meanings mentioned above and in particular the meanings mentioned as being preferred (compounds I-B26). Examples of these are the compounds I-B26.1 to I-B26.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methylphenyl, R⁴ is chlorine, R⁵ is hydrogen and X, Y, R¹ and R² have the meanings mentioned above and in particular the meanings mentioned as being preferred (compounds I-B27). Examples of these are the compounds I-B27.1 to I-B27.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methoxycarbonylphenyl, R⁴ is chlorine, R⁵ is hydrogen and X, Y, R¹ and R² have the meanings mentioned above and in particular the meanings mentioned as being preferred (compounds I-B28). Examples of these are the compounds I-B28.1 to I-B28.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-4-fluorophenyl, R⁴ is chlorine, R⁵ is hydrogen and X, Y, R¹ and R² have the meanings mentioned above and in particular the meanings mentioned as being preferred (compounds I-B29). Examples of these are the compounds I-B29.1 to I-B29.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-5-fluorophenyl, R⁴ is chlorine, R⁵ is hydrogen and X, Y, R¹ and R² have the meanings mentioned above and in particular the meanings mentioned as being preferred (compounds I-B30). Examples of these are the compounds I-B30.1 to I-B30.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-5-chlorophenyl, R⁴ is chlorine, R⁵ is hydrogen and X, Y, R¹ and R² have the meanings mentioned above and in particular the meanings mentioned as being preferred (compounds I-B31). Examples of these are the compounds I-B31.1 to I-B31.414 in which X—R² and Y—R¹ together have in each case the meanings given in one row of Table A.

Particularly preferred compounds of the formula I are the pyrazolopyrimidines of the formula I-B in which R³ is 2-fluoro-6-chlorophenyl, R⁴ is methyl, R⁵ is hydrogen and X, Y, R¹ and R² have the meanings mentioned above and in particular the meanings mentioned as being preferred (compounds I-B32). Examples of these are the compounds I-B32.1 to I-B32.414 in which X—R² and Y—R¹ together have in each case the meanings given in one row of Table A.

Particularly preferred compounds of the formula I are also the pyrazolopyrimidines of the formula I-B in which R³ is 2,6-difluorophenyl, R⁴ is methyl, R⁵ is hydrogen and X, Y, R¹ and R² have the meanings mentioned above and in particular the meanings mentioned as being preferred (compounds I-B33). Examples of these are the compounds I-B33.1 to I-B33.414 in which X—R² and Y—R¹ together have in each case the meanings given in one row of Table A.

Particularly preferred compounds of the formula I are also the pyrazolopyrimidines of the formula I-B in which R³ is 2,6-dichlorophenyl, R⁴ is methyl, R⁵ is hydrogen and X, Y, R¹ and R² have the meanings mentioned above and in particular the meanings mentioned as being preferred (compounds I-B34). Examples of these are the compounds II-B34.1 to I-B34.414 in which X—R² and Y—R¹ together have in each case the meanings given in one row of Table A.

Particularly preferred compounds of the formula I are also the pyrazolopyrimidines of the formula I-B in which R³ is 2-fluoro-6-methylphenyl, R⁴ is methyl, R⁵ is hydrogen and X, Y, R¹ and R² have the meanings mentioned above and in particular the meanings mentioned as being preferred (compounds I-B35). Examples of these are the compounds I-B35.1 to I-B35.414 in which X—R² and Y—R¹ together have in each case the meanings given in one row of Table A.

Particularly preferred compounds of the formula I are also the pyrazolopyrimidines of the formula I-B in which R³ is 2,4,6-trifluorophenyl, R⁴ is methyl, R⁵ is hydrogen and X, Y, R¹ and R² have the meanings mentioned above and in particular the meanings mentioned as being preferred (compounds I-B36). Examples of these are the compounds I-B36.1 to I-B36.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methoxyphenyl, R⁴ is methyl, R⁵ is hydrogen and X, Y, R¹ and R² have the meanings mentioned above and in particular the meanings mentioned as being preferred (compounds I-B37). Examples of these are the compounds I-B37.1 to I-B37.414 in which X—R² and Y—R¹ together have in each case the meanings given in one row of Table A.

Particularly preferred compounds of the formula I are also the pyrazolopyrimidines of the formula I-B in which R³ is 2-chloro-6-methylphenyl, R⁴ is methyl, R⁵ is hydrogen and X, Y, R¹ and R² have the meanings mentioned above and in particular the meanings mentioned as being preferred (compounds I-B38). Examples of these are the compounds I-B38.1 to I-B38.414 in which X—R² and Y—R¹ together have in each case the meanings given in one row of Table A.

Particularly preferred compounds of the formula I are also the pyrazolopyrimidines of the formula I-B in which R³ is pentafluorophenyl, R⁴ is methyl, R⁵ is hydrogen and X, Y, R¹ and R² have the meanings mentioned above and in particular the meanings mentioned as being preferred (compounds I-B39). Examples of these are the compounds I-B39.1 to I-B39.414 in which X—R² and Y—R¹ together have in each case the meanings given in one row of Table A.

Particularly preferred compounds of the formula I are also the pyrazolopyrimidines of the formula I-B in which R³ is 2-methyl-4-fluorophenyl, R⁴ is methyl, R⁵ is hydrogen and X, Y, R¹ and R² have the meanings mentioned above and in particular the meanings mentioned as being preferred (compounds I-B40). Examples of these are the compounds I-B40.1 to I-B40.414 in which X—R² and Y—R¹ together have in each case the meanings given in one row of Table A.

Particularly preferred compounds of the formula I are also the pyrazolopyrimidines of the formula I-B in which R³ is 2-trifluoromethylphenyl, R⁴ is methyl, R⁵ is hydrogen and X, Y, R¹ and R² have the meanings mentioned above and in particular the meanings mentioned as being preferred (compounds I-B41). Examples of these are the compounds I-B41.1 to I-B41.414 in which X—R² and Y—R¹ together have in each case the meanings given in one row of Table A.

Particularly preferred compounds of the formula I are also the pyrazolopyrimidines of the formula I-B in which R³ is 2-methoxy-6-fluorophenyl, R⁴ is methyl, R⁵ is hydrogen and X, Y, R¹ and R² have the meanings mentioned above and in particular the meanings mentioned as being preferred (compounds I-B42). Examples of these are the compounds I-B42.1 to I-B42.414 in which X—R² and Y—R¹ together have in each case the meanings given in one row of Table A.

Particularly preferred compounds of the formula I are also the pyrazolopyrimidines of the formula I-B in which R³ is 2-chlorophenyl, R⁴ is methyl, R⁵ is hydrogen and X, Y, R¹ and R² have the meanings mentioned above and in particular the meanings mentioned as being preferred (compounds I-B43). Examples of these are the compounds I-B43.1 to I-B43.414 in which X—R² and Y—R¹ together have in each case the meanings given in one row of Table A.

Particularly preferred compounds of the formula I are also the pyrazolopyrimidines of the formula I-B in which R³ is 2-fluorophenyl, R⁴ is methyl, R⁵ is hydrogen and X, Y, R¹ and R² have the meanings mentioned above and in particular the meanings mentioned as being preferred (compounds I-B44). Examples of these are the compounds I-B44.1 to I-B44.414 in which X—R² and Y—R¹ together have in each case the meanings given in one row of Table A.

Particularly preferred compounds of the formula I are also the pyrazolopyrimidines of the formula I-B in which R³ is 2,4-difluorophenyl, R⁴ is methyl, R⁵ is hydrogen and X, Y, R¹ and R² have the meanings mentioned above and in particular the meanings mentioned as being preferred (compounds I-B45). Examples of these are the compounds I-B45.1 to I-B45.414 in which X—R² and Y—R¹ together have in each case the meanings given in one row of Table A.

Particularly preferred compounds of the formula I are also the pyrazolopyrimidines of the formula I-B in which R³ is 2-fluoro-4-chlorophenyl, R⁴ is methyl, R⁵ is hydrogen and X, Y, R¹ and R² have the meanings mentioned above and in particular the meanings mentioned as being preferred (compounds I-B46). Examples of these are the compounds I-B46.1 to I-B46.414 in which X—R² and Y—R¹ together have in each case the meanings given in one row of Table A.

Particularly preferred compounds of the formula I are also the pyrazolopyrimidines of the formula I-B in which R³ is 4-fluoro-6-chlorophenyl, R⁴ is methyl, R⁵ is hydrogen and X, Y, R¹ and R² have the meanings mentioned above and in particular the meanings mentioned as being preferred (compounds I-B47). Examples of these are the compounds I-B47.1 to I-B47.414 in which X—R² and Y—R¹ together have in each case the meanings given in one row of Table A.

Particularly preferred compounds of the formula I are also the pyrazolopyrimidines of the formula I-B in which R³ is 2,3-difluorophenyl, R⁴ is methyl, R⁵ is hydrogen and X, Y, R¹ and R² have the meanings mentioned above and in particular the meanings mentioned as being preferred (compounds I-B48). Examples of these are the compounds I-B48.1 to I-B48.414 in which X—R² and Y—R¹ together have in each case the meanings given in one row of Table A.

Particularly preferred compounds of the formula I are also the pyrazolopyrimidines of the formula I-B in which R³ is 2,5-difluorophenyl, R⁴ is methyl, R⁵ is hydrogen and X, Y, R¹ and R² have the meanings mentioned above and in particular the meanings mentioned as being preferred (compounds I-B49). Examples of these are the compounds I-B49.1 to I-B49.414 in which X—R² and Y—R¹ together have in each case the meanings given in one row of Table A.

Particularly preferred compounds of the formula I are also the pyrazolopyrimidines of the formula I-B in which R³ is 2,3,4-trifluorophenyl, R⁴ is methyl, R⁵ is hydrogen and X, Y, R¹ and R² have the meanings mentioned above and in particular the meanings mentioned as being preferred (compounds I-B50). Examples of these are the compounds I-B50.1 to I-B50.414 in which X—R² and Y—R¹ together have in each case the meanings given in one row of Table A.

Particularly preferred compounds of the formula I are also the pyrazolopyrimidines of the formula I-B in which R³ is 2-methylphenyl, R⁴ is methyl, R⁵ is hydrogen and X, Y, R¹ and R² have the meanings mentioned above and in particular the meanings mentioned as being preferred (compounds I-Bs). Examples of these are the compounds I-B51.1 to I-B51.414 in which X—R² and Y—R¹ together have in each case the meanings given in one row of Table A.

Particularly preferred compounds of the formula I are also the pyrazolopyrimidines of the formula I-B in which R³ is 2,4-dimethylphenyl, R⁴ is methyl, R⁵ is hydrogen and X, Y, R¹ and R² have the meanings mentioned above and in particular the meanings mentioned as being preferred (compounds I-B51). Examples of these are the compounds I-B51.1 to I-B51.414 in which X—R² and Y—R¹ together have in each case the meanings given in one row of Table A.

Particularly preferred compounds of the formula I are also the pyrazolopyrimidines of the formula I-B in which R³ is 2-methyl-4-chlorophenyl, R⁴ is methyl, R⁵ is hydrogen and X, Y, R¹ and R² have the meanings mentioned above and in particular the meanings mentioned as being preferred (compounds I-B52). Examples of these are the compounds I-B52.1 to I-B52.414 in which X—R² and Y—R¹ together have in each case the meanings given in one row of Table A.

Particularly preferred compounds of the formula I are also the pyrazolopyrimidines of the formula I-B in which R³ is 2-fluoro-4-methylphenyl, R⁴ is methyl, R⁵ is hydrogen and X, Y, R¹ and R² have the meanings mentioned above and in particular the meanings mentioned as being preferred (compounds I-B53). Examples of these are the compounds I-B53.1 to I-B53.414 in which X—R² and Y—R¹ together have in each case the meanings given in one row of Table A.

Particularly preferred compounds of the formula I are also the pyrazolopyrimidines of the formula I-B in which R³ is 2,6-dimethylphenyl, R⁴ is methyl, R⁵ is hydrogen and X, Y, R¹ and R² have the meanings mentioned above and in particular the meanings mentioned as being preferred (compounds I-B54). Examples of these are the compounds I-B54.1 to I-B54.414 in which X—R² and Y—R¹ together have in each case the meanings given in one row of Table A.

Particularly preferred compounds of the formula I are also the pyrazolopyrimidines of the formula I-B in which R³ is 2,4,5-trimethylphenyl, R⁴ is methyl, R⁵ is hydrogen and X, Y, R¹ and R² have the meanings mentioned above and in particular the meanings mentioned as being preferred (compounds I-B55). Examples of these are the compounds I-B55.1 to I-B55.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-cyanophenyl, R⁴ is methyl, R⁵ is hydrogen and X, Y, R¹ and R² have the meanings mentioned above and in particular the meanings mentioned as being preferred (compounds I-B56). Examples of these are the compounds I-B56.1 to I-B56.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methylphenyl, R⁴ is methyl, R⁵ is hydrogen and X, Y, R¹ and R² have the meanings mentioned above and in particular the meanings mentioned as being preferred (compounds I-B57). Examples of these are the compounds I-B57.1 to I-B57.414 in which X—R² and Y—R¹ together have in each case the meanings given in one row of Table A.

Particularly preferred compounds of the formula I are also the pyrazolopyrimidines of the formula I-B in which R³ is 2,6-difluoro-4-methoxycarbonylphenyl, R⁴ is methyl, R⁵ is hydrogen and X, Y, R¹ and R² have the meanings mentioned above and in particular the meanings mentioned as being preferred (compounds I-B58). Examples of these are the compounds I-B58.1 to I-B58.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-4-fluorophenyl, R⁴ is methyl, R⁵ is hydrogen and X, Y, R¹ and R² have the meanings mentioned above and in particular the meanings mentioned as being preferred (compounds I-B59). Examples of these are the compounds I-B59.1 to I-B59.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-5-fluorophenyl, R⁴ is methyl, R⁵ is hydrogen and X, Y, R¹ and R² have the meanings mentioned above and in particular the meanings mentioned as being preferred (compounds I-B60). Examples of these are the compounds I-B60.1 to I-B60.414 in which X—R² and Y—R¹ together have in each case the meanings given in one row of Table A.

Particularly preferred compounds of the formula I are also the pyrazolopyrimidines of the formula I-B in which R³ is 2-trifluoromethyl-5-chlorophenyl, R⁴ is methyl, R⁵ is hydrogen and X, Y, R¹ and R² have the meanings mentioned above and in particular the meanings mentioned as being preferred (compounds I-B61). Examples of these are the compounds I-B61.1 to I-B61.414 in which X—R² and Y—R¹ together have in each case the meanings given in one row of Table A.

TABLE A

| No. | Y—R¹ | X—R² |
|---|---|---|
| 1 | H | H |
| 2 | CH₃ | H |
| 3 | CH₃ | CH₃ |
| 4 | CH₃ | CH₂CH₃ |
| 5 | CH₃ | Cl |
| 6 | CH₃ | OCH₃ |
| 7 | CH₃ | OC₂H₅ |
| 8 | CH₃ | N(CH₃)₂ |
| 9 | CH₃ | N(CH₃)C₂H₅ |
| 10 | CH₃ | N(CH₃)C(O)CH₃ |
| 11 | CH₂CH₃ | H |
| 12 | CH₂CH₃ | CH₃ |
| 13 | CH₂CH₃ | CH₂CH₃ |
| 14 | CH₂CH₃ | Cl |
| 15 | CH₂CH₃ | OCH₃ |
| 16 | CH₂CH₃ | OC₂H₅ |
| 17 | CH₂CH₃ | N(CH₃)₂ |
| 18 | CH₂CH₃ | N(CH₃)C₂H₅ |
| 19 | CH₂CH₃ | N(CH₃)C(O)CH₃ |
| 20 | CH₂CF₃ | H |
| 21 | CH₂CF₃ | CH₃ |
| 22 | CH₂CF₃ | CH₂CH₃ |
| 23 | CH₂CF₃ | Cl |
| 24 | CH₂CF₃ | OCH₃ |
| 25 | CH₂CF₃ | OC₂H₅ |
| 26 | CH₂CF₃ | N(CH₃)₂ |
| 27 | CH₂CF₃ | N(CH₃)C₂H₅ |
| 28 | CH₂CF₃ | N(CH₃)C(O)CH₃ |
| 29 | CH₂CCl₃ | H |
| 30 | CH₂CCl₃ | CH₃ |
| 31 | CH₂CCl₃ | CH₂CH₃ |
| 32 | CH₂CCl₃ | Cl |
| 33 | CH₂CCl₃ | OCH₃ |
| 34 | CH₂CCl₃ | OC₂H₅ |
| 35 | CH₂CCl₃ | N(CH₃)₂ |
| 36 | CH₂CCl₃ | N(CH₃)C₂H₅ |
| 37 | CH₂CCl₃ | N(CH₃)C(O)CH₃ |
| 38 | CH₂CH₂CH₃ | H |
| 39 | CH₂CH₂CH₃ | CH₃ |
| 40 | CH₂CH₂CH₃ | CH₂CH₃ |
| 41 | CH₂CH₂CH₃ | CH₂CH₂CH₃ |
| 42 | CH₂CH₂CH₃ | Cl |
| 43 | CH₂CH₂CH₃ | OCH₃ |
| 44 | CH₂CH₂CH₃ | OC₂H₅ |
| 45 | CH₂CH₂CH₃ | N(CH₃)₂ |
| 46 | CH₂CH₂CH₃ | N(CH₃)C₂H₅ |
| 47 | CH₂CH₂CH₃ | N(CH₃)C(O)CH₃ |
| 48 | CH(CH₃)₂ | H |
| 49 | CH(CH₃)₂ | CH₃ |
| 50 | CH(CH₃)₂ | CH₂CH₃ |
| 51 | CH(CH₃)₂ | Cl |
| 52 | CH(CH₃)₂ | OCH₃ |
| 53 | CH(CH₃)₂ | OC₂H₅ |
| 54 | CH(CH₃)₂ | N(CH₃)₂ |
| 55 | CH(CH₃)₂ | N(CH₃)C₂H₅ |
| 56 | CH(CH₃)₂ | N(CH₃)C(O)CH₃ |
| 57 | (±)CH(CH₃)—CH₂CH₃ | H |
| 58 | (±)CH(CH₃)—CH₂CH₃ | CH₃ |
| 59 | (±)CH(CH₃)—CH₂CH₃ | CH₂CH₃ |
| 60 | (±)CH(CH₃)—CH₂CH₃ | Cl |
| 61 | (±)CH(CH₃)—CH₂CH₃ | OCH₃ |
| 62 | (±)CH(CH₃)—CH₂CH₃ | OC₂H₅ |
| 63 | (±)CH(CH₃)—CH₂CH₃ | N(CH₃)₂ |
| 64 | (±)CH(CH₃)—CH₂CH₃ | N(CH₃)C₂H₅ |
| 65 | (±)CH(CH₃)—CH₂CH₃ | N(CH₃)C(O)CH₃ |
| 66 | (S)CH(CH₃)—CH₂CH₃ | H |
| 67 | (S)CH(CH₃)—CH₂CH₃ | CH₃ |
| 68 | (S)CH(CH₃)—CH₂CH₃ | CH₂CH₃ |
| 69 | (S)CH(CH₃)—CH₂CH₃ | Cl |
| 70 | (S)CH(CH₃)—CH₂CH₃ | OCH₃ |
| 71 | (S)CH(CH₃)—CH₂CH₃ | OC₂H₅ |
| 72 | (S)CH(CH₃)—CH₂CH₃ | N(CH₃)₂ |
| 73 | (S)CH(CH₃)—CH₂CH₃ | N(CH₃)C₂H₅ |
| 74 | (S)CH(CH₃)—CH₂CH₃ | N(CH₃)C(O)CH₃ |
| 75 | (R)CH(CH₃)—CH₂CH₃ | H |
| 76 | (R)CH(CH₃)—CH₂CH₃ | CH₃ |
| 77 | (R)CH(CH₃)—CH₂CH₃ | CH₂CH₃ |
| 78 | (R)CH(CH₃)—CH₂CH₃ | Cl |
| 79 | (R)CH(CH₃)—CH₂CH₃ | OCH₃ |
| 80 | (R)CH(CH₃)—CH₂CH₃ | OC₂H₅ |
| 81 | (R)CH(CH₃)—CH₂CH₃ | N(CH₃)₂ |
| 82 | (R)CH(CH₃)—CH₂CH₃ | N(CH₃)C₂H₅ |
| 83 | (R)CH(CH₃)—CH₂CH₃ | N(CH₃)C(O)CH₃ |
| 84 | (±)CH(CH₃)—CH(CH₃)₂ | H |
| 85 | (±)CH(CH₃)—CH(CH₃)₂ | CH₃ |
| 86 | (±)CH(CH₃)—CH(CH₃)₂ | CH₂CH₃ |
| 87 | (±)CH(CH₃)—CH(CH₃)₂ | Cl |
| 88 | (±)CH(CH₃)—CH(CH₃)₂ | OCH₃ |
| 89 | (±)CH(CH₃)—CH(CH₃)₂ | OC₂H₅ |
| 90 | (±)CH(CH₃)—CH(CH₃)₂ | N(CH₃)₂ |
| 91 | (±)CH(CH₃)—CH(CH₃)₂ | N(CH₃)C₂H₅ |
| 92 | (±)CH(CH₃)—CH(CH₃)₂ | N(CH₃)C(O)CH₃ |
| 93 | (S)CH(CH₃)—CH(CH₃)₂ | H |
| 94 | (S)CH(CH₃)—CH(CH₃)₂ | CH₃ |
| 95 | (S)CH(CH₃)—CH(CH₃)₂ | CH₂CH₃ |
| 96 | (S)CH(CH₃)—CH(CH₃)₂ | Cl |
| 97 | (S)CH(CH₃)—CH(CH₃)₂ | OCH₃ |
| 98 | (S)CH(CH₃)—CH(CH₃)₂ | OC₂H₅ |
| 99 | (S)CH(CH₃)—CH(CH₃)₂ | N(CH₃)₂ |
| 100 | (S)CH(CH₃)—CH(CH₃)₂ | N(CH₃)C₂H₅ |
| 101 | (S)CH(CH₃)—CH(CH₃)₂ | N(CH₃)C(O)CH₃ |
| 102 | (R)CH(CH₃)—CH(CH₃)₂ | H |
| 103 | (R)CH(CH₃)—CH(CH₃)₂ | CH₃ |
| 104 | (R)CH(CH₃)—CH(CH₃)₂ | CH₂CH₃ |
| 105 | (R)CH(CH₃)—CH(CH₃)₂ | Cl |
| 106 | (R)CH(CH₃)—CH(CH₃)₂ | OCH₃ |
| 107 | (R)CH(CH₃)—CH(CH₃)₂ | OC₂H₅ |
| 108 | (R)CH(CH₃)—CH(CH₃)₂ | N(CH₃)₂ |
| 109 | (R)CH(CH₃)—CH(CH₃)₂ | N(CH₃)C₂H₅ |
| 110 | (R)CH(CH₃)—CH(CH₃)₂ | N(CH₃)C(O)CH₃ |
| 111 | (±)CH(CH₃)—C(CH₃)₃ | H |
| 112 | (±)CH(CH₃)—C(CH₃)₃ | CH₃ |
| 113 | (±)CH(CH₃)—C(CH₃)₃ | CH₂CH₃ |
| 114 | (±)CH(CH₃)—C(CH₃)₃ | Cl |
| 115 | (±)CH(CH₃)—C(CH₃)₃ | OCH₃ |
| 116 | (±)CH(CH₃)—C(CH₃)₃ | OC₂H₅ |
| 117 | (±)CH(CH₃)—C(CH₃)₃ | N(CH₃)₂ |
| 118 | (±)CH(CH₃)—C(CH₃)₃ | N(CH₃)C₂H₅ |
| 119 | (±)CH(CH₃)—C(CH₃)₃ | N(CH₃)C(O)CH₃ |
| 120 | (S)CH(CH₃)—C(CH₃)₃ | H |
| 121 | (S)CH(CH₃)—C(CH₃)₃ | CH₃ |
| 122 | (S)CH(CH₃)—C(CH₃)₃ | CH₂CH₃ |
| 123 | (S)CH(CH₃)—C(CH₃)₃ | Cl |
| 124 | (S)CH(CH₃)—C(CH₃)₃ | OCH₃ |
| 125 | (S)CH(CH₃)—C(CH₃)₃ | OC₂H₅ |
| 126 | (S)CH(CH₃)—C(CH₃)₃ | N(CH₃)₂ |
| 127 | (S)CH(CH₃)—C(CH₃)₃ | N(CH₃)C₂H₅ |
| 128 | (S)CH(CH₃)—C(CH₃)₃ | N(CH₃)C(O)CH₃ |
| 129 | (R)CH(CH₃)—C(CH₃)₃ | H |
| 130 | (R)CH(CH₃)—C(CH₃)₃ | CH₃ |
| 131 | (R)CH(CH₃)—C(CH₃)₃ | CH₂CH₃ |
| 132 | (R)CH(CH₃)—C(CH₃)₃ | Cl |
| 133 | (R)CH(CH₃)—C(CH₃)₃ | OCH₃ |
| 134 | (R)CH(CH₃)—C(CH₃)₃ | OC₂H₅ |
| 135 | (R)CH(CH₃)—C(CH₃)₃ | N(CH₃)₂ |
| 136 | (R)CH(CH₃)—C(CH₃)₃ | N(CH₃)C₂H₅ |
| 137 | (R)CH(CH₃)—C(CH₃)₃ | N(CH₃)C(O)CH₃ |
| 138 | (±)CH(CH₃)—CF₃ | H |
| 139 | (±)CH(CH₃)—CF₃ | CH₃ |
| 140 | (±)CH(CH₃)—CF₃ | CH₂CH₃ |
| 141 | (±)CH(CH₃)—CF₃ | Cl |
| 142 | (±)CH(CH₃)—CF₃ | OCH₃ |
| 143 | (±)CH(CH₃)—CF₃ | OC₂H₅ |
| 144 | (±)CH(CH₃)—CF₃ | N(CH₃)₂ |
| 145 | (±)CH(CH₃)—CF₃ | N(CH₃)C₂H₅ |
| 146 | (±)CH(CH₃)—CF₃ | N(CH₃)C(O)CH₃ |
| 147 | (S)CH(CH₃)—CF₃ | H |
| 148 | (S)CH(CH₃)—CF₃ | CH₃ |
| 149 | (S)CH(CH₃)—CF₃ | CH₂CH₃ |
| 150 | (S)CH(CH₃)—CF₃ | Cl |
| 151 | (S)CH(CH₃)—CF₃ | OCH₃ |
| 152 | (S)CH(CH₃)—CF₃ | OC₂H₅ |
| 153 | (S)CH(CH₃)—CF₃ | N(CH₃)₂ |
| 154 | (S)CH(CH₃)—CF₃ | N(CH₃)C₂H₅ |
| 155 | (S)CH(CH₃)—CF₃ | N(CH₃)C(O)CH₃ |
| 156 | (R)CH(CH₃)—CF₃ | H |
| 157 | (R)CH(CH₃)—CF₃ | CH₃ |
| 158 | (R)CH(CH₃)—CF₃ | CH₂CH₃ |
| 159 | (R)CH(CH₃)—CF₃ | Cl |
| 160 | (R)CH(CH₃)—CF₃ | OCH₃ |
| 161 | (R)CH(CH₃)—CF₃ | OC₂H₅ |
| 162 | (R)CH(CH₃)—CF₃ | N(CH₃)₂ |
| 163 | (R)CH(CH₃)—CF₃ | N(CH₃)C₂H₅ |
| 164 | (R)CH(CH₃)—CF₃ | N(CH₃)C(O)CH₃ |
| 165 | (±)CH(CH₃)—CCl₃ | H |
| 166 | (±)CH(CH₃)—CCl₃ | CH₃ |
| 167 | (±)CH(CH₃)—CCl₃ | CH₂CH₃ |
| 168 | (±)CH(CH₃)—CCl₃ | Cl |
| 169 | (±)CH(CH₃)—CCl₃ | OCH₃ |
| 170 | (±)CH(CH₃)—CCl₃ | OC₂H₅ |
| 171 | (±)CH(CH₃)—CCl₃ | N(CH₃)₂ |
| 172 | (±)CH(CH₃)—CCl₃ | N(CH₃)C₂H₅ |
| 173 | (±)CH(CH₃)—CCl₃ | N(CH₃)C(O)CH₃ |
| 174 | (S)CH(CH₃)—CCl₃ | H |
| 175 | (S)CH(CH₃)—CCl₃ | CH₃ |
| 176 | (S)CH(CH₃)—CCl₃ | CH₂CH₃ |
| 177 | (S)CH(CH₃)—CCl₃ | Cl |
| 178 | (S)CH(CH₃)—CCl₃ | OCH₃ |
| 179 | (S)CH(CH₃)—CCl₃ | OC₂H₅ |
| 180 | (S)CH(CH₃)—CCl₃ | N(CH₃)₂ |
| 181 | (S)CH(CH₃)—CCl₃ | N(CH₃)C₂H₅ |
| 182 | (S)CH(CH₃)—CCl₃ | N(CH₃)C(O)CH₃ |
| 183 | (R)CH(CH₃)—CCl₃ | H |
| 184 | (R)CH(CH₃)—CCl₃ | CH₃ |
| 185 | (R)CH(CH₃)—CCl₃ | CH₂CH₃ |
| 186 | (R)CH(CH₃)—CCl₃ | Cl |
| 187 | (R)CH(CH₃)—CCl₃ | OCH₃ |
| 188 | (R)CH(CH₃)—CCl₃ | OC₂H₅ |
| 189 | (R)CH(CH₃)—CCl₃ | N(CH₃)₂ |
| 190 | (R)CH(CH₃)—CCl₃ | N(CH₃)C₂H₅ |
| 191 | (R)CH(CH₃)—CCl₃ | N(CH₃)C(O)CH₃ |
| 192 | CH₂CF₂CF₃ | H |
| 193 | CH₂CF₂CF₃ | CH₃ |
| 194 | CH₂CF₂CF₃ | CH₂CH₃ |
| 195 | CH₂CF₂CF₃ | Cl |
| 196 | CH₂CF₂CF₃ | OCH₃ |
| 197 | CH₂CF₂CF₃ | OC₂H₅ |
| 198 | CH₂CF₂CF₃ | N(CH₃)₂ |
| 199 | CH₂CF₂CF₃ | N(CH₃)C₂H₅ |
| 200 | CH₂CF₂CF₃ | N(CH₃)C(O)CH₃ |
| 201 | CH₂(CF₂)₂CF₃ | H |
| 202 | CH₂(CF₂)₂CF₃ | CH₃ |
| 203 | CH₂(CF₂)₂CF₃ | CH₂CH₃ |
| 204 | CH₂(CF₂)₂CF₃ | Cl |
| 205 | CH₂(CF₂)₂CF₃ | OCH₃ |
| 206 | CH₂(CF₂)₂CF₃ | OC₂H₅ |
| 207 | CH₂(CF₂)₂CF₃ | N(CH₃)₂ |
| 208 | CH₂(CF₂)₂CF₃ | N(CH₃)C₂H₅ |
| 209 | CH₂(CF₂)₂CF₃ | N(CH₃)C(O)CH₃ |
| 210 | CH₂C(CH₃)═CH₂ | H |
| 211 | CH₂C(CH₃)═CH₂ | CH₃ |
| 212 | CH₂C(CH₃)═CH₂ | CH₂CH₃ |
| 213 | CH₂C(CH₃)═CH₂ | Cl |
| 214 | CH₂C(CH₃)═CH₂ | OCH₃ |
| 215 | CH₂C(CH₃)═CH₂ | OC₂H₅ |
| 216 | CH₂C(CH₃)═CH₂ | N(CH₃)₂ |
| 217 | CH₂C(CH₃)═CH₂ | N(CH₃)C₂H₅ |
| 218 | CH₂C(CH₃)═CH₂ | N(CH₃)C(O)CH₃ |
| 219 | CH₂CH═CH₂ | H |
| 220 | CH₂CH═CH₂ | CH₃ |
| 221 | CH₂CH═CH₂ | CH₂CH₃ |
| 222 | CH₂CH═CH₂ | Cl |
| 223 | CH₂CH═CH₂ | OCH₃ |
| 224 | CH₂CH═CH₂ | OC₂H₅ |
| 225 | CH₂CH═CH₂ | N(CH₃)₂ |
| 226 | CH₂CH═CH₂ | N(CH₃)C₂H₅ |
| 227 | CH₂CH═CH₂ | N(CH₃)C(O)CH₃ |
| 228 | CH(CH₃)CH═CH₂ | H |
| 229 | CH(CH₃)CH═CH₂ | CH₃ |
| 230 | CH(CH₃)CH═CH₂ | CH₂CH₃ |
| 231 | CH(CH₃)CH═CH₂ | Cl |
| 232 | CH(CH₃)CH═CH₂ | OCH₃ |
| 233 | CH(CH₃)CH═CH₂ | OC₂H₅ |
| 234 | CH(CH₃)CH═CH₂ | N(CH₃)₂ |
| 235 | CH(CH₃)CH═CH₂ | N(CH₃)C₂H₅ |
| 236 | CH(CH₃)CH═CH₂ | N(CH₃)C(O)CH₃ |
| 237 | CH(CH₃)C(CH₃)═CH₂ | H |
| 238 | CH(CH₃)C(CH₃)═CH₂ | CH₃ |
| 239 | CH(CH₃)C(CH₃)═CH₂ | CH₂CH₃ |
| 240 | CH(CH₃)C(CH₃)═CH₂ | Cl |
| 241 | CH(CH₃)C(CH₃)═CH₂ | OCH₃ |
| 242 | CH(CH₃)C(CH₃)═CH₂ | OC₂H₅ |
| 243 | CH(CH₃)C(CH₃)═CH₂ | N(CH₃)₂ |
| 244 | CH(CH₃)C(CH₃)═CH₂ | N(CH₃)C₂H₅ |
| 245 | CH(CH₃)C(CH₃)═CH₂ | N(CH₃)C(O)CH₃ |
| 246 | cyclopentyl | H |
| 247 | cyclopentyl | CH₃ |
| 248 | cyclopentyl | CH₂CH₃ |
| 249 | cyclopentyl | Cl |
| 250 | cyclopentyl | OCH₃ |
| 251 | cyclopentyl | OC₂H₅ |
| 252 | cyclopentyl | N(CH₃)₂ |
| 253 | cyclopentyl | N(CH₃)C₂H₅ |
| 254 | cyclopentyl | N(CH₃)C(O)CH₃ |
| 255 | cyclohexyl | H |
| 256 | cyclohexyl | CH₃ |
| 257 | cyclohexyl | CH₂CH₃ |
| 258 | cyclohexyl | Cl |
| 259 | cyclohexyl | OCH₃ |
| 260 | cyclohexyl | OC₂H₅ |
| 261 | cyclohexyl | N(CH₃)₂ |
| 262 | cyclohexyl | N(CH₃)C₂H₅ |
| 263 | cyclohexyl | N(CH₃)C(O)CH₃ |
| 264 | CF₃ | H |
| 265 | CF₃ | CH₃ |
| 266 | CF₃ | CH₂CH₃ |
| 267 | CF₃ | Cl |
| 268 | CF₃ | OCH₃ |
| 269 | CF₃ | OC₂H₅ |
| 270 | CF₃ | N(CH₃)₂ |
| 271 | CF₃ | N(CH₃)C₂H₅ |
| 272 | CF₃ | N(CH₃)C(O)CH₃ |
| 273 | CCl₃ | H |
| 274 | CCl₃ | CH₃ |
| 275 | CCl₃ | CH₂CH₃ |
| 276 | CCl₃ | Cl |
| 277 | CCl₃ | OCH₃ |
| 278 | CCl₃ | OC₂H₅ |
| 279 | CCl₃ | N(CH₃)₂ |
| 280 | CCl₃ | N(CH₃)C₂H₅ |
| 281 | CCl₃ | N(CH₃)C(O)CH₃ |
| 282 | CF₂CF₃ | H |
| 283 | CF₂CF₃ | CH₃ |
| 284 | CF₂CF₃ | CH₂CH₃ |
| 285 | CF₂CF₃ | Cl |
| 286 | CF₂CF₃ | OCH₃ |
| 287 | CF₂CF₃ | OC₂H₅ |
| 288 | CF₂CF₃ | N(CH₃)₂ |
| 289 | CF₂CF₃ | N(CH₃)C₂H₅ |
| 290 | CF₂CF₃ | N(CH₃)C(O)CH₃ |
| 291 | (CF₂)₂CF₃ | H |
| 292 | (CF₂)₂CF₃ | CH₃ |
| 293 | (CF₂)₂CF₃ | CH₂CH₃ |
| 294 | (CF₂)₂CF₃ | Cl |
| 295 | (CF₂)₂CF₃ | OCH₃ |
| 296 | (CF₂)₂CF₃ | OC₂H₅ |
| 297 | (CF₂)₂CF₃ | N(CH₃)₂ |
| 298 | (CF₂)₂CF₃ | N(CH₃)C₂H₅ |
| 299 | (CF₂)₂CF₃ | N(CH₃)C(O)CH₃ |
| 300 | C(CH₃)═CH₂ | H |
| 301 | C(CH₃)═CH₂ | CH₃ |
| 302 | C(CH₃)═CH₂ | CH₂CH₃ |
| 303 | C(CH₃)═CH₂ | Cl |
| 304 | C(CH₃)═CH₂ | OCH₃ |
| 305 | C(CH₃)═CH₂ | OC₂H₅ |
| 306 | C(CH₃)═CH₂ | N(CH₃)₂ |
| 307 | C(CH₃)═CH₂ | N(CH₃)C₂H₅ |
| 308 | C(CH₃)═CH₂ | N(CH₃)C(O)CH₃ |
| 309 | CH═CH₂ | H |
| 310 | CH═CH₂ | CH₃ |
| 311 | CH═CH₂ | CH₂CH₃ |
| 312 | CH═CH₂ | Cl |
| 313 | CH═CH₂ | OCH₃ |
| 314 | CH═CH₂ | OC₂H₅ |
| 315 | CH═CH₂ | N(CH₃)₂ |
| 316 | CH═CH₂ | N(CH₃)C₂H₅ |
| 317 | CH═CH₂ | N(CH₃)C(O)CH₃ |
| 318 | phenyl | H |
| 319 | phenyl | CH₃ |
| 320 | phenyl | CH₂CH₃ |
| 321 | phenyl | Cl |
| 322 | phenyl | OCH₃ |
| 323 | phenyl | OC₂H₅ |
| 324 | phenyl | N(CH₃)₂ |
| 325 | phenyl | N(CH₃)C₂H₅ |
| 326 | phenyl | N(CH₃)C(O)CH₃ |
| 327 | CH₂phenyl | H |
| 328 | CH₂phenyl | CH₃ |
| 329 | CH₂phenyl | CH₂CH₃ |
| 330 | CH₂phenyl | Cl |
| 331 | CH₂phenyl | OCH₃ |
| 332 | CH₂phenyl | OC₂H₅ |
| 333 | CH₂phenyl | N(CH₃)₂ |
| 334 | CH₂phenyl | N(CH₃)C₂H₅ |
| 335 | CH₂phenyl | N(CH₃)C(O)CH₃ |

| | |
|---|---|
| 336 | —(CH₂)₂CH═CHCH₂— |
| 337 | —(CH₂)₂C(CH₃)═CHCH₂— |
| 338 | —(CH₂)₂CH(CH₃)(CH₂)₂— |
| 339 | —(CH₂)₂CHF(CH₂)₂— |
| 340 | —(CH₂)₃CHFCH₂— |
| 341 | —(CH₂)₂CH(CF₃)(CH₂)₂— |
| 342 | —(CH₂)₂O(CH₂)₂— |
| 343 | —(CH₂)₂S(CH₂)₂— |
| 344 | —(CH₂)₅— |
| 345 | —(CH₂)₆— |
| 346 | —(CH₂)₄— |
| 347 | —CH₂CH═CHCH₂— |
| 348 | —CH(CH₃)(CH₂)₃— |
| 349 | —CH₂CH(CH₃)(CH₂)₂— |

| | | |
|---|---|---|
| 350 | 1-piperidinyl | H |
| 351 | 1-piperidinyl | CH₃ |
| 352 | 1-piperidinyl | CH₂CH₃ |
| 353 | 1-pyrrolidinyl | H |
| 354 | 1-pyrrolidinyl | CH₃ |
| 355 | 1-pyrrolidinyl | CH₂CH₃ |
| 356 | morpholin-4-yl | H |
| 357 | morpholin-4-yl | CH₃ |
| 358 | morpholin-4-yl | CH₂CH₃ |
| 359 | 2,5-dihydropyrrol-1-yl | H |
| 360 | 2,5-dihydropyrrol-1-yl | CH₃ |
| 361 | 2,5-dihydropyrrol-1-yl | CH₂CH₃ |
| 362 | H | Cl |
| 363 | H | OCH₃ |
| 364 | H | OC₂H₅ |
| 365 | H | N(CH₃)₂ |
| 366 | H | N(CH₃)C₂H₅ |
| 367 | H | N(CH₃)C(O)CH₃ |
| 368 | H | CH(CH₃)₂ |
| 369 | H | CH₂CH(CH₃)₂ |
| 370 | H | CH(CH₃)CH₂CH₃ |
| 371 | H | C(CH₃)₃ |
| 372 | CH₃ | CH(CH₃)₂ |
| 373 | CH₃ | CH₂CH(CH₃)₂ |
| 374 | CH₃ | CH(CH₃)CH₂CH₃ |
| 375 | CH₃ | C(CH₃)₃ |
| 376 | CH₂CH₃ | CH₂CH(CH₃)₂ |
| 377 | CH₂CH₃ | CH(CH₃)CH₂CH₃ |
| 378 | CH₂CH₃ | C(CH₃)₃ |
| 379 | CH₂CF₃ | CH(CH₃)₂ |
| 380 | CH₂CF₃ | CH₂CH(CH₃)₂ |
| 381 | CH₂CF₃ | CH(CH₃)CH₂CH₃ |
| 382 | CH₂CF₃ | C(CH₃)₃ |
| 383 | CH₂CH₂CH₃ | CH(CH₃)₂ |
| 384 | CH₂CH₂CH₃ | CH₂CH(CH₃)₂ |
| 385 | CH₂CH₂CH₃ | CH(CH₃)CH₂CH₃ |
| 386 | CH₂CH₂CH₃ | C(CH₃)₃ |
| 387 | CH(CH₃)₂ | CH(CH₃)₂ |
| 388 | CH(CH₃)₂ | CH₂CH(CH₃)₂ |
| 389 | CH(CH₃)₂ | CH(CH₃)CH₂CH₃ |
| 390 | CH(CH₃)₂ | C(CH₃)₃ |
| 391 | (±)CH(CH₃)—CF₃ | CH(CH₃)₂ |
| 392 | (±)CH(CH₃)—CF₃ | CH₂CH(CH₃)₂ |
| 393 | (±)CH(CH₃)—CF₃ | CH(CH₃)CH₂CH₃ |
| 394 | (±)CH(CH₃)—CF₃ | C(CH₃)₃ |
| 395 | (S)(CH(CH₃)—CF₃ | CH(CH₃)₂ |
| 396 | (S)(CH(CH₃)—CF₃ | CH₂CH(CH₃)₂ |
| 397 | (S)(CH(CH₃)—CF₃ | CH(CH₃)CH₂CH₃ |
| 398 | (S)(CH(CH₃)—CF₃ | C(CH₃)₃ |
| 399 | CH₂CF₂CF₃ | CH(CH₃)₂ |
| 400 | CH₂CF₂CF₃ | CH₂CH(CH₃)₂ |
| 401 | CH₂CF₂CF₃ | CH(CH₃)CH₂CH₃ |
| 402 | CH₂CF₂CF₃ | C(CH₃)₃ |
| 403 | CF₃ | CH(CH₃)₂ |
| 404 | CF₃ | CH₂CH(CH₃)₂ |
| 405 | CF₃ | CH(CH₃)CH₂CH₃ |
| 406 | CF₃ | C(CH₃)₃ |
| 407 | CF₂CF₃ | CH(CH₃)₂ |
| 408 | CF₂CF₃ | CH₂CH(CH₃)₂ |
| 409 | CF₂CF₃ | CH(CH₃)CH₂CH₃ |
| 410 | CF₂CF₃ | C(CH₃)₃ |
| 411 | (CF₂)₂CF₃ | CH(CH₃)₂ |
| 412 | (CF₂)₂CF₃ | CH₂CH(CH₃)₂ |
| 413 | (CF₂)₂CF₃ | CH(CH₃)CH₂CH₃ |
| 414 | (CF₂)₂CF₃ | C(CH₃)₃ |

Further preferred embodiments of the invention relate to tautomers of the formula II. Among the tautomers of the formula II, preference is given to those compounds in which W^{a} is O or S. In the tautomers of the formula II, V is preferably a chemical bond. With respect to preferred meanings of the variables R³, R⁴, R⁵ and A, what was stated above for formula I applies. Preferred radicals R²⁰ are those which are indicated in formula I as preferred radicals for R¹ or R². In particular, R²⁰ is a radical of the formula C or B as indicated for R¹ or R².

Preferred tautomers II are in particular the compounds of the formulae II-A and II-B 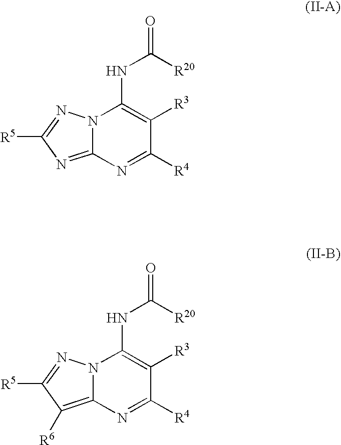
in which R³, R⁴, R⁵, R⁶ and R²⁰ have the meanings indicated above.

Particularly preferred among these are the compounds of the formulae II-A and II-B in which R³ is 2-fluoro-6-chlorophenyl, R⁴ is chlorine, R⁵ is hydrogen, R⁶ is hydrogen and R²⁰ has the meanings mentioned above and in particular the meanings mentioned as being preferred (compounds II-A1 and II-B1). Examples of these are the compounds II-A1.1 to II-A1.39 and II-B1.1 to II-B1.39 in which R²⁰ has the meaning given in one row of Table B.

Particularly preferred among these are also the compounds of the formulae II-A and II-B in which R³ is 2,6-difluorophenyl, R⁴ is chlorine, R⁵ is hydrogen, R⁶ is hydrogen and R²⁰ has the meanings mentioned above and in particular the meanings mentioned as being preferred (compounds II-A2 and II-B2). Examples of these are the compounds II-A2.1 to II-A2.39 and II-B2.1 to II-B2.39 in which R²⁰ has the meaning given in one row of Table B.

Particularly preferred among these are furthermore the compounds of the formulae II-A and II-B in which R³ is 2,6-dichlorophenyl, R⁴ is chlorine, R⁵ is hydrogen, R⁶ is hydrogen and R²⁰ has the meanings mentioned above and in particular the meanings mentioned as being preferred (compounds II-A3 and II-B3). Examples of these are the compounds II-A3.1 to II-A3.39 and II-B3.1 to II-B3.39 in which R²⁰ has the meaning given in one row of Table B.

Particularly preferred among these are also the compounds of the formulae II-A and II-B in which R³ is 2-fluoro-6-methylphenyl, R⁴ is chlorine, R⁵ is hydrogen, R⁶ is hydrogen and R²⁰ has the meanings mentioned above and in particular the meanings mentioned as being preferred (compounds II-A4 and II-B4). Examples of these are the compounds II-A4.1 to II-A4.39 and II-B4.1 to II-B4.39 in which R²⁰ has the meaning given in one row of Table B.

Particularly preferred among these are also the compounds of the formulae II-A and II-B in which R³ is 2,4,6-trifluorophenyl, R⁴ is chlorine, R⁵ is hydrogen, R⁶ is hydrogen and R²⁰ has the meanings mentioned above and in particular the meanings mentioned as being preferred (compounds II-A5 and II-B5). Examples of these are the compounds II-A5.1 to II-A5.39 and II-B5.1 to II-B5.39 in which R²⁰ has the meaning given in one row of Table B.

Particularly preferred among these are also the compounds of the formulae II-A and II-B in which R³ is 2,6-difluoro-4-methoxyphenyl, R⁴ is chlorine, R⁵ is hydrogen, R⁶ is hydrogen and R²⁰ has the meanings mentioned above and in particular the meanings mentioned as being preferred (compounds II-A6 and II-B6). Examples of these are the compounds II-A6.1 to II-A6.39 and II-B6.1 to II-B6.39 in which R²⁰ has the meaning given in one row of Table B.

Particularly preferred among these are also the compounds of the formulae II-A and II-B in which R³ is 2-chloro-6-methylphenyl, R⁴ is chlorine, R⁵ is hydrogen, R⁶ is hydrogen and R²⁰ has the meanings mentioned above and in particular the meanings mentioned as being preferred (compounds II-A7 and II-B7). Examples of these are the compounds II-A7.1 to II-A7.39 and II-B7.1 to II-B7.39 in which R²⁰ has the meaning given in one row of Table B.

Particularly preferred among these are also the compounds of the formulae II-A and II-B in which R³ is pentafluorophenyl, R⁴ is chlorine, R⁵ is hydrogen, R⁵ is hydrogen and R²⁰ has the meanings mentioned above and in particular the meanings mentioned as being preferred (compounds II-A8 and II-B8). Examples of these are the compounds II-A8.1 to II-A8.39 and II-B8.1 to II-B8.39 in which R²⁰ has the meaning given in one row of Table B.

Particularly preferred among these are also the compounds of the formulae II-A and II-B in which R³ is 2-methyl-4-fluorophenyl, R⁴ is chlorine, R⁵ is hydrogen, R⁶ is hydrogen and R²⁰ has the meanings mentioned above and in particular the meanings mentioned as being preferred (compounds II-A9 and II-B9). Examples of these are the compounds II-A9.1 to II-A9.39 and II-B9.1 to II-B9.39 in which R²⁰ has the meaning given in one row of Table B.

Particularly preferred among these are also the compounds of the formulae II-A and II-B in which R³ is 2-trifluoromethylphenyl, R⁴ is chlorine, R⁵ is hydrogen, R⁶ is hydrogen and R²⁰ has the meanings mentioned above and in particular the meanings mentioned as being preferred (compounds II-A10 and II-B10). Examples of these are the compounds II-A10.1 to II-A10.39 and II-B10.1 to II-B10.39 in which R²⁰ has the meaning given in one row of Table B.

Particularly preferred among these are also the compounds of the formulae II-A and II-B in which R³ is 2-methoxy-6-fluorophenyl, R⁴ is chlorine, R⁵ is hydrogen, R⁶ is hydrogen and R²⁰ has the meanings mentioned above and in particular the meanings mentioned as being preferred (compounds II-A11 and II-B11). Examples of these are the compounds II-A11.1 to II-A11.39 and II-B11.1 to II-B11.39 in which R²⁰ has the meaning given in one row of Table B.

Particularly preferred among these are also the compounds of the formulae II-A and II-B in which R³ is 2-chlorophenyl, R⁴ is chlorine, R⁵ is hydrogen, R⁶ is hydrogen and R²⁰ has the meanings mentioned above and in particular the meanings mentioned as being preferred (compounds II-A12 and II-B12). Examples of these are the compounds II-A12.1 to II-A12.39 and II-B12.1 to II-B12.39 in which R²⁰ has the meaning given in one row of Table B.

Particularly preferred among these are also the compounds of the formulae II-A and II-B in which R³ is 2-fluorophenyl, R⁴ is chlorine, R⁵ is hydrogen, R⁶ is hydrogen and R²⁰ has the meanings mentioned above and in particular the meanings mentioned as being preferred (compounds II-A13 and II-B13). Examples of these are the compounds II-A13.1 to II-A13.39 and II-B13.1 to II-B13.39 in which R²⁰ has the meaning given in one row of Table B.

Particularly preferred among these are also the compounds of the formulae II-A and II-B in which R³ is 2,4-difluorophenyl, R⁴ is chlorine, R⁵ is hydrogen, R⁶ is hydrogen and R²⁰ has the meanings mentioned above and in particular the meanings mentioned as being preferred (compounds II-A14 and II-B14). Examples of these are the compounds II-A14.1 to II-A14.39 and II-B14.1 to II-B14.39 in which R²⁰ has the meaning given in one row of Table B.

Particularly preferred among these are also the compounds of the formulae II-A and II-B in which R³ is 2-fluoro-4-chlorophenyl, R⁴ is chlorine, R⁵ is hydrogen, R⁶ is hydrogen and R²⁰ has the meanings mentioned above and in particular the meanings mentioned as being preferred (compounds II-A15 and II-B15). Examples of these are the compounds II-A15.1 to II-A15.39 and II-B15.1 to II-B15.39 in which R²⁰ has the meaning given in one row of Table B.

Particularly preferred among these are also the compounds of the formulae II-A and II-B in which R³ is 4-fluoro-2-chlorophenyl, R⁴ is chlorine, R⁵ is hydrogen, R⁶ is hydrogen and R²⁰ has the meanings mentioned above and in particular the meanings mentioned as being preferred (compounds II-A16 and II-B16). Examples of these are the compounds II-A16.1 to II-A16.39 and II-B16.1 to II-B16.39 in which R²⁰ has the meaning given in one row of Table B.

Particularly preferred among these are also the compounds of the formulae II-A and II-B in which R³ is 2,3-difluorophenyl, R⁴ is chlorine, R⁵ is hydrogen, R⁶ is hydrogen and R²⁰ has the meanings mentioned above and in particular the meanings mentioned as being preferred (compounds II-A17 and II-B17). Examples of these are the compounds II-A17.1 to II-A17.39 and II-B17.1 to II-B17.39 in which R²⁰ has the meaning given in one row of Table B.

Particularly preferred among these are also the compounds of the formulae II-A and II-B in which R³ is 2,5-difluorophenyl, R⁴ is chlorine, R⁵ is hydrogen, R⁶ is hydrogen and R²⁰ has the meanings mentioned above and in particular the meanings mentioned as being preferred (compounds II-A18 and II-B18). Examples of these are the compounds II-A18.1 to II-A18.39 and II-B18.1 to II-B18.39 in which R²⁰ has the meaning given in one row of Table B.

Particularly preferred among these are also the compounds of the formulae II-A and II-B in which R³ is 2,3,4-trifluorophenyl, R⁴ is chlorine, R⁵ is hydrogen, R⁶ is hydrogen and R²⁰ has the meanings mentioned above and in particular the meanings mentioned as being preferred (compounds II-A19 and II-B19). Examples of these are the compounds II-A19.1 to II-A19.39 and II-B19.1 to II-B19.39 in which R²⁰ has the meaning given in one row of Table B.

Particularly preferred among these are also the compounds of the formulae II-A and II-B in which R³ is 2-methylphenyl, R⁴ is chlorine, R⁵ is hydrogen, R⁶ is hydrogen and R²⁰ has the meanings mentioned above and in particular the meanings mentioned as being preferred (compounds II-A20 and II-B20). Examples of these are the compounds II-A20.1 to II-A20.39 and II-B20.1 to II-B20.39 in which R²⁰ has the meaning given in one row of Table B.

Particularly preferred among these are also the compounds of the formulae II-A and II-B in which R³ is 2,4-dimethylphenyl, R⁴ is chlorine, R⁵ is hydrogen, R⁶ is hydrogen and R²⁰ has the meanings mentioned above and in particular the meanings mentioned as being preferred (compounds II-A21 and II-B21). Examples of these are the compounds II-A21.1 to II-A21.39 and II-B21.1 to II-B21.39 in which R²⁰ has the meaning given in one row of Table B.

Particularly preferred among these are also the compounds of the formulae II-A and II-B in which R³ is 2-methyl-4-chlorophenyl, R⁴ is chlorine, R⁵ is hydrogen, R⁶ is hydrogen and R²⁰ has the meanings mentioned above and in particular the meanings mentioned as being preferred (compounds II-A22 and II-B22). Examples of these are the compounds II-A22.1 to II-A22.39 and II-B22.1 to II-B22.39 in which R²⁰ has the meaning given in one row of Table B.

Particularly preferred among these are also the compounds of the formulae II-A and II-B in which R³ is 2-fluoro-4-methylphenyl, R⁴ is chlorine, R⁵ is hydrogen, R⁶ is hydrogen and R²⁰ has the meanings mentioned above and in particular the meanings mentioned as being preferred (compounds II-A23 and II-B23). Examples of these are the compounds II-A23.1 to II-A23.39 and II-B23.1 to II-B23.39 in which R²⁰ has the meaning given in one row of Table B.

Particularly preferred among these are also the compounds of the formulae II-A and II-B in which R³ is 2,6-dimethylphenyl, R⁴ is chlorine, R⁵ is hydrogen, R⁶ is hydrogen and R²⁰ has the meanings mentioned above and in particular the meanings mentioned as being preferred (compounds II-A24 and II-B24). Examples of these are the compounds II-A24.1 to II-A24.39 and II-B24.1 to II-B24.39 in which R²⁰ has the meaning given in one row of Table B.

Particularly preferred among these are also the compounds of the formulae II-A and II-B in which R³ is 2,4,5-trimethylphenyl, R⁴ is chlorine, R⁵ is hydrogen, R⁶ is hydrogen and R²⁰ has the meanings mentioned above and in particular the meanings mentioned as being preferred (compounds II-A25 and II-B25). Examples of these are the compounds II-A25.1 to II-A25.39 and II-B25.1 to II-B25.39 in which R²⁰ has the meaning given in one row of Table B.

Particularly preferred among these are also the compounds of the formulae II-A and II-B in which R³ is 2,6-difluoro-4-cyanophenyl, R⁴ is chlorine, R⁵ is hydrogen, R⁶ is hydrogen and R²⁰ has the meanings mentioned above and in particular the meanings mentioned as being preferred (compounds II-A26 and II-B26). Examples of these are the compounds II-A26.1 to II-A26.39 and II-B26.1 to II-B26.39 in which R²⁰ has the meaning given in one row of Table B.

Particularly preferred among these are also the compounds of the formulae II-A and II-B in which R³ is 2,6-difluoro-4-methylphenyl, R⁴ is chlorine, R⁵ is hydrogen, R⁶ is hydrogen and R²⁰ has the meanings mentioned above and in particular the meanings mentioned as being preferred (compounds II-A27 and II-B27). Examples of these are the compounds II-A27.1 to II-A27.39 and II-B27.1 to II-B27.39 in which R²⁰ has the meaning given in one row of Table B.

Particularly preferred among these are also the compounds of the formulae II-A and II-B in which R³ is 2,6-difluoro-4-methoxycarbonylphenyl, R⁴ is chlorine, R⁵ is hydrogen, R⁶ is hydrogen and R²⁰ has the meanings mentioned above and in particular the meanings mentioned as being preferred (compounds II-A28 and II-B28). Examples of these are the compounds II-A28.1 to II-A28.39 and II-B28.1 to II-B28.39 in which R²⁰ has the meaning given in one row of Table B.

Particularly preferred among these are also the compounds of the formulae II-A and II-B in which R³ is 2-trifluoromethyl-4-fluorophenyl, R⁴ is chlorine, R⁵ is hydrogen, R⁶ is hydrogen and R²⁰ has the meanings mentioned above and in particular the meanings mentioned as being preferred (compounds II-29 and II-B29). Examples of these are the compounds I-A29.1 to II-A29.39 and II-B29.1 to II-B29.39 in which R²⁰ has the meaning given in one row of Table B.

Particularly preferred among these are the compounds of the formulae II-A and II-B in which R³ is 2-trifluoromethyl-5-fluorophenyl, R⁴ is chlorine, R⁵ is hydrogen, R⁶ is hydrogen and R²⁰ has the meanings mentioned above and in particular the meanings mentioned as being preferred (compounds II-A30 and II-B30). Examples of these are the compounds II-A30.1 to II-A30.39 and II-B30.1 to II-B30.39 in which R²⁰ has the meaning given in one row of Table B.

Particularly preferred among these are the compounds of the formulae II-A and II-B in which R³ is 2-trifluoromethyl-5-chlorophenyl, R⁴ is chlorine, R⁵ is hydrogen, R⁶ is hydrogen and R²⁰ has the meanings mentioned above and in particular the meanings mentioned as being preferred (compounds II-A31 and II-B31). Examples of these are the compounds II-A31.1 to II-A31.39 and II-B31.1 to II-B31.39 in which R²⁰ has the meaning given in one row of Table B.

Particularly preferred among these are furthermore the compounds of the formulae II-A and II-B in which R³ is 2-fluoro-6-chlorophenyl, R⁴ is methyl, R⁵ is hydrogen, R⁶ is hydrogen and R²⁰ has the meanings mentioned above and in particular the meanings mentioned as being preferred (compounds II-A32 and II-B32). Examples of these are the compounds II-A32.1 to II-A32.39 and II-B32.1 to II-B32.39 in which R²⁰ has the meaning given in one row of Table B.

Particularly preferred among these are also the compounds of the formulae II-A and II-B in which R³ is 2,6-difluorophenyl, R⁴ is methyl, R⁵ is hydrogen, R⁶ is hydrogen and R²⁰ has the meanings mentioned above and in particular the meanings mentioned as being preferred (compounds II-A33 and II-B33). Examples of these are the compounds II-A33.1 to II-A33.39 and II-B33.1 to II-B33.39 in which R²⁰ has the meaning given in one row of Table B.

Particularly preferred among these are furthermore the compounds of the formulae II-A and II-B in which R³ is 2,6-dichlorophenyl, R⁴ is methyl, R⁵ is hydrogen, R⁶ is hydrogen and R²⁰ has the meanings mentioned above and in particular the meanings mentioned as being preferred (compounds II-A34 and II-B34). Examples of these are the compounds II-A34.1 to II-A34.39 and II-B34.1 to II-B34.39 in which R²⁰ has the meaning given in one row of Table B.

Particularly preferred among these are also the compounds of the formulae II-A and II-B in which R³ is 2-fluoro-6-methylphenyl, R⁴ is methyl, R⁵ is hydrogen, R⁵ is hydrogen and R²⁰ has the meanings mentioned above and in particular the meanings mentioned as being preferred (compounds II-A35 and II-B35). Examples of these are the compounds II-A35.1 to II-A35.39 and II-B35.1 to II-B35.39 in which R²⁰ has the meaning given in one row of Table B.

Particularly preferred among these are also the compounds of the formulae II-A and II-B in which R³ is 2,4,6-trifluorophenyl, R⁴ is methyl, R⁵ is hydrogen, R⁶ is hydrogen and R²⁰ has the meanings mentioned above and in particular the meanings mentioned as being preferred (compounds II-A36 and II-B36). Examples of these are the compounds II-A36.1 to II-A36.39 and II-B36.1 to II-B36.39 in which R²⁰ has the meaning given in one row of Table B.

Particularly preferred among these are also the compounds of the formulae II-A and II-B in which R³ is 2,6-difluoro-4-methoxyphenyl, R⁴ is methyl, R⁵ is hydrogen, R⁶ is hydrogen and R²⁰ has the meanings mentioned above and in particular the meanings mentioned as being preferred (compounds II-A37 and II-B37). Examples of these are the compounds II-A37.1 to II-A37.39 and II-B37.1 to II-B37.39 in which R²⁰ has the meaning given in one row of Table B.

Particularly preferred among these are also the compounds of the formulae II-A and II-B in which R³ is 2-chloro-6-methylphenyl, R⁴ is methyl, R⁵ is hydrogen, R⁶ is hydrogen and R²⁰ has the meanings mentioned above and in particular the meanings mentioned as being preferred (compounds II-A38 and II-B38). Examples of these are the compounds II-A38.1 to II-A38.39 and II-B38.1 to II-B38.39 in which R²⁰ has the meaning given in one row of Table B.

Particularly preferred among these are also the compounds of the formulae I-A and II-B in which R³ is 2-methyl-4-fluorophenyl, R⁴ is methyl, R⁵ is hydrogen, R⁶ is hydrogen and R²⁰ has the meanings mentioned above and in particular the meanings mentioned as being preferred (compounds II-A39 and II-B39). Examples of these are the compounds II-A39.1 to II-A39.39 and II-B39.1 to II-B39.39 in which R²⁰ has the meaning given in one row of Table B.

Particularly preferred among these are also the compounds of the formulae II-A and II-B in which R³ is pentafluorophenyl, R⁴ is methyl, R⁵ is hydrogen, R⁶ is hydrogen and R²⁰ has the meanings mentioned above and in particular the meanings mentioned as being preferred (compounds II-A40 and II-B40). Examples of these are the compounds II-A40.1 to II-A40.39 and II-B40.1 to II-B40.39 in which R²⁰ has the meaning given in one row of Table B.

Particularly preferred among these are also the compounds of the formulae II-A and II-B in which R³ is 2-trifluoromethylphenyl, R⁴ is methyl, R⁵ is hydrogen, R⁶ is hydrogen and R²⁰ has the meanings mentioned above and in particular the meanings mentioned as being preferred (compounds II-A41 and II-B41). Examples of these are the compounds I-A41.1 to II-A41.39 and II-B41.1 to II-B41.39 in which R²⁰ has the meaning given in one row of Table B.

Particularly preferred among these are also the compounds of the formulae II-A and II-B in which R³ is 2-methoxy-6-fluorophenyl, R⁴ is methyl, R⁵ is hydrogen, R⁶ is hydrogen and R²⁰ has the meanings mentioned above and in particular the meanings mentioned as being preferred (compounds II-A42 and II-B42). Examples of these are the compounds II-A42.1 to II-A42.39 and II-B42.1 to II-B42.39 in which R²⁰ has the meaning given in one row of Table B.

Particularly preferred among these are also the compounds of the formulae II-A and II-B in which R³ is 2-chlorophenyl, R⁴ is methyl, R⁵ is hydrogen, R⁵ is hydrogen and R²⁰ has the meanings mentioned above and in particular the meanings mentioned as being preferred (compounds II-A43 and II-B43). Examples of these are the compounds II-A43.1 to II-A43.39 and II-B43.1 to II-B43.39 in which R²⁰ has the meaning given in one row of Table B.

Particularly preferred among these are also the compounds of the formulae II-A and II-B in which R³ is 2-fluorophenyl, R⁴ is methyl, R⁵ is hydrogen, R⁶ is hydrogen and R²⁰ has the meanings mentioned above and in particular the meanings mentioned as being preferred (compounds II-A44 and II-B44). Examples of these are the compounds II-A44.1 to II-A44.39 and II-B44.1 to II-B44.39 in which R²⁰ has the meaning given in one row of Table B.

Particularly preferred among these are also the compounds of the formulae II-A and II-B in which R³ is 2,4-difluorophenyl, R⁴ is methyl, R⁵ is hydrogen, R⁶ is hydrogen and R²⁰ has the meanings mentioned above and in particular the meanings mentioned as being preferred (compounds II-A45 and II-B45). Examples of these are the compounds II-A45.1 to II-A45.39 and II-B45.1 to II-B45.39 in which R²⁰ has the meaning given in one row of Table B.

Particularly preferred among these are also the compounds of the formulae II-A and II-B in which R³ is 2-fluoro-4-chlorophenyl, R⁴ is methyl, R⁵ is hydrogen, R⁵ is hydrogen and R²⁰ has the meanings mentioned above and in particular the meanings mentioned as being preferred (compounds II-46 and II-B46). Examples of these are the compounds II-A46.1 to II-A46.39 and II-B46.1 to II-B46.39 in which R²⁰ has the meaning given in one row of Table B.

Particularly preferred among these are also the compounds of the formulae II-A and II-B in which R³ is 4-fluoro-2-chlorophenyl, R⁴ is methyl, R⁵ is hydrogen, R⁶ is hydrogen and R²⁰ has the meanings mentioned above and in particular the meanings mentioned as being preferred (compounds II-A47 and II-B47). Examples of these are the compounds II-A47.1 to II-A47.39 and II-B47.1 to II-B47.39 in which R²⁰ has the meaning given in one row of Table B.

Particularly preferred among these are also the compounds of the formulae II-A and II-B in which R³ is 2,3-difluorophenyl, R⁴ is methyl, R⁵ is hydrogen, R⁵ is hydrogen and R²⁰ has the meanings mentioned above and in particular the meanings mentioned as being preferred (compounds II-A48 and II-B48). Examples of these are the compounds II-A48.1 to II-A48.39 and II-B48.1 to II-B48.39 in which R²⁰ has the meaning given in one row of Table B.

Particularly preferred among these are also the compounds of the formulae II-A and II-B in which R³ is 2,5-difluorophenyl, R⁴ is methyl, R⁵ is hydrogen, R⁶ is hydrogen and R²⁰ has the meanings mentioned above and in particular the meanings mentioned as being preferred (compounds II-A49 and II-B49). Examples of these are the compounds II-A49.1 to II-A49.39 and II-B49.1 to II-B49.39 in which R²⁰ has the meaning given in one row of Table B.

Particularly preferred among these are also the compounds of the formulae II-A and II-B in which R³ is 2,3,4-trifluorophenyl, R⁴ is methyl, R⁵ is hydrogen, R⁶ is hydrogen and R²⁰ has the meanings mentioned above and in particular the meanings mentioned as being preferred (compounds II-A50 and II-B50). Examples of these are the compounds II-A50.1 to II-A50.39 and II-B50.1 to II-B50.39 in which R²⁰ has the meaning given in one row of Table B.

Particularly preferred among these are also the compounds of the formulae II-A and II-B in which R³ is 2-methylphenyl, R⁴ is methyl, R⁵ is hydrogen, R⁶ is hydrogen and R²⁰ has the meanings mentioned above and in particular the meanings mentioned as being preferred (compounds II-A51 and II-B51). Examples of these are the compounds II-A51.1 to II-A51.39 and II-B51.1 to II-B51.39 in which R²⁰ has the meaning given in one row of Table B.

Particularly preferred among these are also the compounds of the formulae II-A and II-B in which R³ is 2,4-dimethylphenyl, R⁴ is methyl, R⁵ is hydrogen, R⁶ is hydrogen and R²⁰ has the meanings mentioned above and in particular the meanings mentioned as being preferred (compounds II-A52 and I-B52). Examples of these are the compounds II-A52.1 to II-A52.39 and II-B52.1 to II-B52.39 in which R²⁰ has the meaning given in one row of Table B.

Particularly preferred among these are also the compounds of the formulae II-A and II-B in which R³ is 2-methyl-4-chlorophenyl, R⁴ is methyl, R⁵ is hydrogen, R⁶ is hydrogen and R²⁰ has the meanings mentioned above and in particular the meanings mentioned as being preferred (compounds II-A53 and II-B53). Examples of these are the compounds II-A53.1 to II-A53.39 and II-B53.1 to II-B53.39 in which R²⁰ has the meaning given in one row of Table B.

Particularly preferred among these are also the compounds of the formulae II-A and II-B in which R³ is 2-fluoro-4-methylphenyl, R⁴ is methyl, R⁵ is hydrogen, R⁶ is hydrogen and R²⁰ has the meanings mentioned above and in particular the meanings mentioned as being preferred (compounds II-A54 and II-B54). Examples of these are the compounds II-A54.1 to II-A54.39 and II-B54.1 to II-B54.39 in which R²⁰ has the meaning given in one row of Table B.

Particularly preferred among these are also the compounds of the formulae II-A and II-B in which R³ is 2,6-dimethylphenyl, R⁴ is methyl, R⁵ is hydrogen, R⁶ is hydrogen and R²⁰ has the meanings mentioned above and in particular the meanings mentioned as being preferred (compounds II-A55 and II-B55). Examples of these are the compounds II-A55.1 to II-A55.39 and II-B55.1 to II-B55.39 in which R²⁰ has the meaning given in one row of Table B.

Particularly preferred among these are also the compounds of the formulae II-A and II-B in which R³ is 2,4,5-trimethylphenyl, R⁴ is methyl, R⁵ is hydrogen, R⁶ is hydrogen and R²⁰ has the meanings mentioned above and in particular the meanings mentioned as being preferred (compounds II-A56 and II-B56). Examples of these are the compounds II-A56.1 to II-A56.39 and II-B56.1 to II-B56.39 in which R²⁰ has the meaning given in one row of Table B.

Particularly preferred among these are also the compounds of the formulae II-A and II-B in which R³ is 2,6-difluoro-4-cyanophenyl, R⁴ is methyl, R⁵ is hydrogen, R⁶ is hydrogen and R²⁰ has the meanings mentioned above and in particular the meanings mentioned as being preferred (compounds II-A57 and II-B57). Examples of these are the compounds II-A57.1 to II-A57.39 and II-B57.1 to II-B57.39 in which R²⁰ has the meaning given in one row of Table B.

Particularly preferred among these are also the compounds of the formulae II-A and II-B in which R³ is 2,6-difluoro-4-methylphenyl, R⁴ is methyl, R⁵ is hydrogen, R⁶ is hydrogen and R²⁰ has the meanings mentioned above and in particular the meanings mentioned as being preferred (compounds II-A58 and II-B58). Examples of these are the compounds II-A58.1 to II-A58.39 and II-B58.1 to II-B58.39 in which R²⁰ has the meaning given in one row of Table B.

Particularly preferred among these are also the compounds of the formulae II-A and II-B in which R³ is 2,6-difluoro-4-methoxycarbonylphenyl, R⁴ is methyl, R⁵ is hydrogen, R⁶ is hydrogen and R²⁰ has the meanings mentioned above and in particular the meanings mentioned as being preferred (compounds II-A59 and II-B59). Examples of these are the compounds II-A59.1 to II-A59.39 and II-B59.1 to II-B59.39 in which R²⁰ has the meaning given in one row of Table B.

Particularly preferred among these are also the compounds of the formulae II-A and II-B in which R³ is 2-trifluoromethyl-4-fluorophenyl, R⁴ is methyl, R⁵ is hydrogen, R⁶ is hydrogen and R²⁰ has the meanings mentioned above and in particular the meanings mentioned as being preferred (compounds II-60 and II-B60). Examples of these are the compounds II-A60.1 to II-A60.39 and II-B60.1 to II-B60.39 in which R²⁰ has the meaning given in one row of Table B.

Particularly preferred among these are the compounds of the formulae II-A and II-B in which R³ is 2-trifluoromethyl-5-fluorophenyl, R⁴ is methyl, R⁵ is hydrogen, R⁶ is hydrogen and R²⁰ has the meanings mentioned above and in particular the meanings mentioned as being preferred (compounds II-A61 and II-B61). Examples of these are the compounds II-A61.1 to II-A61.39 and II-B61.1 to II-B61.39 in which R²⁰ has the meaning given in one row of Table B.

Particularly preferred among these are the compounds of the formulae II-A and II-B in which R³ is 2-trifluoromethyl-5-chlorophenyl, R⁴ is methyl, R⁵ is hydrogen, R⁶ is hydrogen and R²⁰ has the meanings mentioned above and in particular the meanings mentioned as being preferred (compounds II-A62 and II-B62). Examples of these are the compounds II-A62.1 to II-A62.39 and II-B62.1 to II-B62.39 in which R²⁰ has the meaning given in one row of Table B.

TABLE B

| No. | R²⁰ |
|---|---|
| 1 | H |
| 2 | CH₃ |
| 3 | CH₂CH₃ |
| 4 | CH₂CF₃ |
| 5 | CH₂CCl₃ |
| 6 | CH₂CH₂CH₃ |
| 7 | CH(CH₃)₂ |
| 8 | (±)CH(CH₃)—CH₂CH₃ |
| 9 | (S)CH(CH₃)—CH₂CH₃ |
| 10 | (R)CH(CH₃)—CH₂CH₃ |
| 11 | (±)CH(CH₃)—CH(CH₃)₂ |
| 12 | (S)CH(CH₃)—CH(CH₃)₂ |
| 13 | (R)CH(CH₃)—CH(CH₃)₂ |
| 14 | (±)CH(CH₃)—C(CH₃)₃ |
| 15 | (S)CH(CH₃)—C(CH₃)₃ |
| 16 | (R)CH(CH₃)—C(CH₃)₃ |
| 17 | (±)CH(CH₃)—CF₃ |
| 18 | (S)CH(CH₃)—CF₃ |
| 19 | (R)CH(CH₃)—CF₃ |
| 20 | (±)CH(CH₃)—CCl₃ |
| 21 | (S)CH(CH₃)—CCl₃ |
| 22 | (R)CH(CH₃)—CCl₃ |
| 23 | CH₂CF₂CF₃ |
| 24 | CH₂(CF₂)₂CF₃ |
| 25 | CH₂C(CH₃)═CH₂ |
| 26 | CH₂CH═CH₂ |
| 27 | CH(CH₃)CH═CH₂ |
| 28 | CH(CH₃)C(CH₃)═CH₂ |
| 29 | cyclopentyl |
| 30 | cyclohexyl |
| 31 | cyclopropyl |
| 32 | CF₃ |
| 33 | CCl₃ |
| 34 | CF₂CF₃ |
| 35 | (CF₂)₂CF₃ |
| 36 | C(CH₃)═CH₂ |
| 37 | CH═CH₂ |
| 38 | phenyl |
| 39 | CH₂phenyl |

The compounds of the formula I according to the invention can be prepared analogously to prior art methods known per se, starting from 7-aminoazolopyrimidines of formula III or 7-haloazolopyrimidines of the formula IV 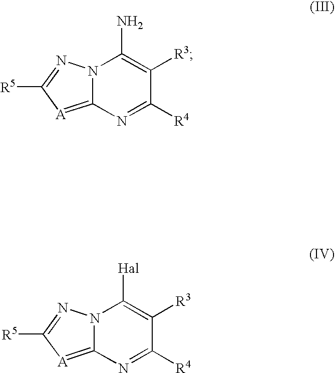
according to the syntheses shown in the schemes below. In the compounds of the formulae III and IV, A, R³, R⁴ and R⁵ have the meanings indicated above. Hal is halogen, in particular chlorine or bromine. The compounds III and IV are known from the prior art cited at the outset or can be prepared analogously to the processes described therein.

Compounds of the formula I in which X and Y are a chemical bond can be prepared, for example, according to the method described by G. A. Grasa et al. J. Org. Chem. 2001, 66(23) pp. 7729-7737 or Stauffer et al., Org. Lett. 2002, 2(10), pp. 1423-1426 by reacting the 7-haloazolopyrimidine IV with an imine of the formula V in the presence of palladium catalysts (see scheme 1)

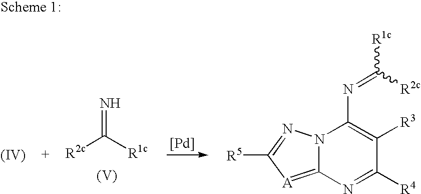

In scheme 1, A, R³, R⁴ and R⁵ have the meanings indicated above. R^{1c} and R^{2c} independently of one another are hydrogen or have the meanings given for R¹ and R², respectively, or R^{1c} and R^{2c} together with the carbon atom, to which they are attached, form a 5-, 6- or 7-membered saturated or unsaturated carbo- or heterocycle, where the latter may have 1, 2, 3 or 4 heteroatoms selected from the group consisting of O, S and N as ring members, where the carbo- and the heterocycle may be partially or fully halogenated or have 1, 2, 3 or 4 of the radicals R⁷ and/or R⁸.

Compounds of the formula I in which X and Y are a chemical bond can furthermore be prepared according to the process shown in scheme 2 from the corresponding 7-aminoazolopyrimidines II. To this end, compound III is initially converted using the method described by Llamas-Saiz et al. (J. Chem. Soc. Perkin Trans. 2, 1991, pp. 1667-1676) into the phosphaimine VI, which can then be converted into the corresponding compound I by reacting either an aldehyde or a ketone VII according to the methods described by Bravo et al. Synlett 1996, p. 887 ff. and Takahashi et al., Synthesis, 1998, pp. 986-990 (see scheme 2):

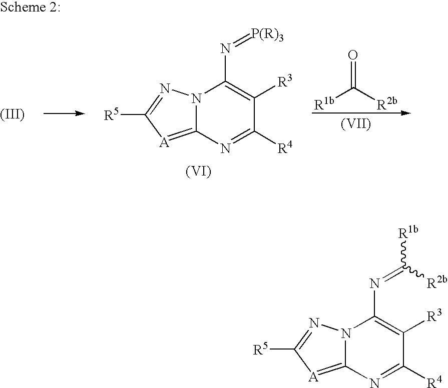

In scheme 2, A, R³, R⁴ and R⁵ are as defined above. R^{1b} and R^{2b} independently of one another are hydrogen or have the meanings given for R¹ and R², respectively, or R^{1b} and R^{2b} together with the carbon atom, to which they are attached, form a 5-, 6- or 7-membered saturated or unsaturated carbo- or heterocycle, where the latter may have 1, 2, 3 or 4 heteroatoms selected from the group consisting of O, S and N as ring members, where the carbo- and the heterocycle may be partially or fully halogenated or have 1, 2, 3 or 4 of the radicals R⁷ and/or R⁸. R is aryl such as phenyl, which is optionally substituted, for example with 1, 2 or 3 substitutents selected from the group consisting of halogen, alkyl and alkoxy.

Compounds of the formula I in which Y—R¹ (or X—R²) is halogen, X (or Y) is a single bond and R² is as defined above may be prepared from the corresponding tautomers of the formula II in which W^{a} is oxygen, R²⁰ corresponds to the radical R² and V is a bond, according to the method described by Stevens et al., J. Am. Chem. Soc. 1953, 75, pp. 657-660 by reaction with a halogenating agent [Hal] (see scheme 3).

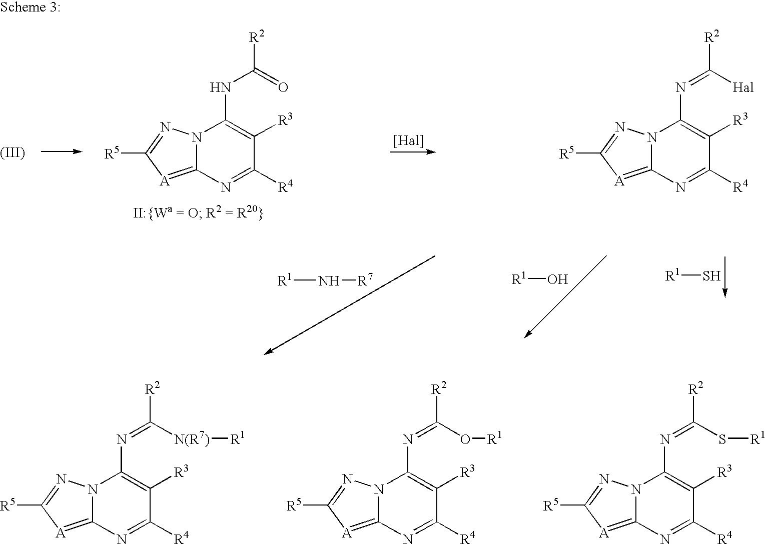

In scheme 3, A, R¹, R³, R⁴, R⁵ and R⁷ are as defined above. Examples of halogenating agents [Hal] are phosphorus halides and sulfur halogen compounds, such as phosphorus oxybromide, phosphorus oxychloride, phosphorus pentachloride, thionyl chloride, thionyl bromide or sulfuryl chloride. The reaction can be carried out in the absence of a solvent or in the presence of a solvent. In one embodiment the reaction is carried out in the presence of a tertiary amine such as triethylamine or pyridine as base. In another preferred embodiment, the reaction is carried out in an aromatic hydrocarbon, such as toluene, in the presence of catalytic amounts of an amide, such as dimethylformamide. Customary reaction temperatures are from −20 to 200° C. or, preferably, from 0 to 160° C.

The halogen compounds I in which Y—R¹ (or X—R²) is halogen can for their part be converted into the corresponding compounds I in which Y is oxygen by reacting them with an alcohol of the formula R¹—OH according to the method described by Stevens et al., J. Am. Chem. Soc. 1953, 75, pp. 657-660. In an analogous manner, the compounds I in which X—R² is halogen afford the compounds I in which X is oxygen. Moreover, in an analogous manner, it is possible to prepare the compounds of the formula I in which X is a bond and Y is a group R⁷ by reaction with secondary amines of the formula R¹—NH—R⁷. Moreover, in an analogous manner, it is possible to prepare the compounds of the formula I in which X is a bond and Y is S by reaction with thioalcohols of the formula R¹—SH (see scheme 3).

Compounds of the formula I in which X is a chemical bond and Y—R¹ is a radical of the formula N(R⁷)R¹ can be prepared from the compounds III by reaction with carboxamide analogs VIII according to the methods described by S. Leistner et al., Pharmazie 1991, 46, pp. 457-458, and Troschutz et al., Arch. Pharm. 1993, 326, 857-864 (see scheme 4). R″ is C₁-C₆-alkyl. Compounds of the formula I in which X is a chemical bond and Y is O can be prepared by reacting III with orthoesters of the formula IX according to the method described by Troschütz et al. Arch. Pharm. 1993, 326, 857-864 (see scheme 4). In scheme 4, A, R¹, R², R³, R⁴, R⁵ and R⁷ are as defined above.

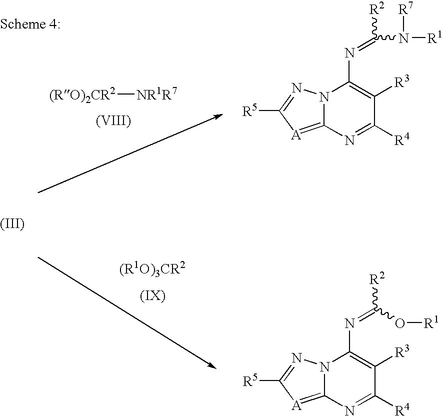

The tautomers of the formula II in which W^{a}═O and V is a chemical bond can be prepared by customary amidation processes from the 7-aminoazolopyrimidines III, for example by reaction with carboxylic acids or carboxylic acid derivatives of the formula R²³—CO-L in which R²³ has one of the meanings given for R²⁰ and L is a nucleophilically replaceable leaving group, for example OH, halogen, in particular chlorine, or the radical of an activated ester group, such as p-nitrophenoxy, if appropriate in the presence of suitable catalysts, auxiliary bases, for example tertiary amines, such as triethylamine or pyridine compounds, and/or dehydrating agents, for example carbodiimides. Methods to achieve this are known from the prior art and can be applied analogously to the preparation of the compounds II where W^{a}═O (see, for example, Werbel et al. J. Heterocycl Chem. 1987, 24, p. 345; Stevens et al. loc.cit., see also J. March, “Advanced Organic Synthesis”, 3^{rd} edition, Wiley & Sons, New York 1985, pp. 370-376 and the literature cited therein). Compounds II where W^{a}═S can be prepared from the compounds II where W^{a}═O by reaction with sulfurizing agents. In an analogous manner, compounds of the formula II in which V is O or S can be prepared by reacting III with derivatives of carbonic acid or thiocarbonic acid, for example chloroformic esters or carbonates. Compounds II in which V is NH can be prepared by reacting III with isocyanates or isothiocyanates.

Compounds of the formula II in which W^{a} is S or O can also be converted into the corresponding compounds I in which X is O or S by using alkylating agents (scheme 5). In scheme 5, A, R¹, R², R³, R⁴, R⁵ and R²⁰ have the meanings given above. W^{a} and X are S or O. Y has the meanings indicated above and is in particular a chemical bond.

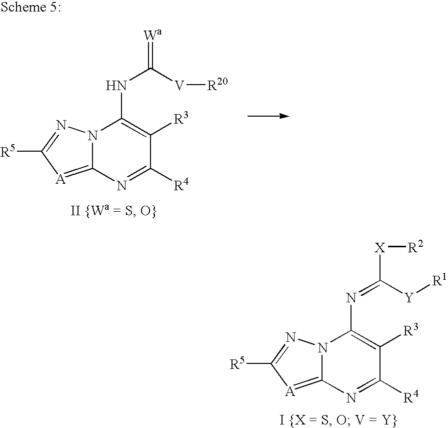

It is furthermore possible to convert compounds of the formula I given below in which Y is a chemical bond and X is oxygen and compounds I in which X—R² is halogen and Y is a chemical bond by reaction with ammonia or a primary amine H₂N—R²¹ into compounds II in which W^{a} is a group NH or NR²¹ and Y—R²⁰ corresponds to the group R¹ (scheme 6). By alkylation with an alkylating agent R⁷-L in which L is a nucleophilically replaceable leaving group, for example halogen, (halo)alkylsulfonate, such as mesylate or triflate, or arylsulfonate, such as tosylate, these compounds can then be converted into the imides I in which Y is a chemical bond and X is a group NR⁷ and R²¹ corresponds to the radical R².

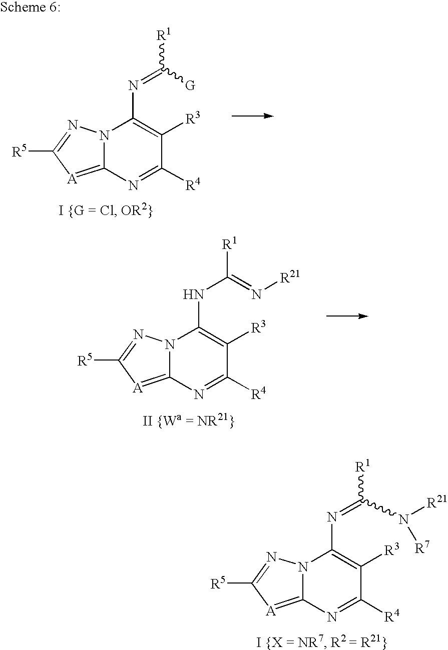

In scheme 6, A, R¹, R², R³, R⁴, R⁵ and R⁷ are as defined above.

The reactions shown in schemes 1 to 6 can be carried out in the absence of a solvent or in solution. Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, or hydrochloric acid or acetic acid. It is also possible to use mixtures of the solvents mentioned.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed from volatile components under reduced pressure and at moderately elevated temperature. If the intermediates and end products are obtained as solids, purification can also be carried out by recrystallization or digestion.

If individual compounds I cannot be obtained by the routes described above, they can be prepared by derivatization of other compounds I.

If the synthesis yields mixtures of isomers, a separation is generally not necessarily required since in some cases the individual isomers can be interconverted during work-up for use or during application (for example under the action of light, acids or bases). Such conversions may also take place after use, for example, in the case of treatment of plants, in the treated plants, or in the harmful fungus to be controlled.

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soya, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally required.

When used in the protection of materials or stored products, the amount of active compound applied depends on the kind of application area and on the desired effect. Amounts customarily applied in the protection of materials are, for example, 0.001 g to 2 kg, preferably 0.005 g to 1 kg, of active compound per cubic meter of treated material.

The compounds I can be converted into the customary formulations, for example solutions, emulsions, suspensions, dusts, powders, pastes and granules. The application form depends on the particular purpose; in each case, it should ensure a fine and uniform distribution of the compound according to the invention.

The formulations are prepared in a known manner, for example by extending the active compound with solvents and/or carriers, if desired using emulsifiers and dispersants. Solvents/auxiliaries which are suitable are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used;
carriers such as ground natural minerals (for example kaolins, clays, talc, chalk) and ground synthetic minerals (for example highly disperse silica, silicates); emulsifiers such as nonionic and anionic emulsifiers (for example polyoxyethylene fatty alcohol ethers, alkylsulfonates and arylsulfonates) and dispersants such as lignosulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

Examples of formulations comprise products for dilution with water, for example

A Water-soluble concentrates (SL)

10 parts by weight of a compound according to the invention are dissolved in water or in a water-soluble solvent. As an alternative, wetters or other auxiliaries are added. The active compound dissolves upon dilution with water;
   B Dispersible concentrates (DC)
20 parts by weight of a compound according to the invention are dissolved in cyclohexanone with addition of a dispersant, for example polyvinylpyrrolidone. Dilution with water gives a dispersion;
   C Emulsifiable concentrates (EC)
15 parts by weight of a compound according to the invention are dissolved in xylene with addition of calcium dodecylbenzenesulfonate and castor oil ethoxylate (in each case 5%). Dilution with water gives an emulsion;
   D Emulsions (EW, EO)
40 parts by weight of a compound according to the invention are dissolved in xylene with addition of calcium dodecylbenzenesulfonate and castor oil ethoxylate (in each case 5%). This mixture is introduced into water by means of an emulsifying machine (Ultraturrax) and made into a homogeneous emulsion. Dilution with water gives an emulsion;
   E Suspensions (SC, OD)
In an agitated ball mill, 20 parts by weight of a compound according to the invention are comminuted with addition of dispersants, wetters and water or an organic solvent to give a fine active compound suspension. Dilution with water gives a stable suspension of the active compound;
   F Water-dispersible granules and water-soluble granules (WG, SG)
50 parts by weight of a compound according to the invention are ground finely with addition of dispersants and wetters and made into water-dispersible or water-soluble granules by means of technical appliances (for example extrusion, spray tower, fluidized bed). Dilution with water gives a stable dispersion or solution of the active compound;
   G Water-dispersible powders and water-soluble powders (WP, SP)
75 parts by weight of a compound according to the invention are ground in a rotor-stator mill with addition of dispersants, wetting agents and silica gel. Dilution with water gives a stable dispersion or solution of the active compound; and products to be applied undiluted, for example
   H Dustable powders (DP)
5 parts by weight of a compound according to the invention are ground finely and mixed intimately with 95% of finely divided kaolin. This gives a dustable product;
   I Granules (GR, FG, GG, MG)
0.5 part by weight of a compound according to the invention is ground finely and associated with 95.5% carriers. Current methods are extrusion, spray-drying or the fluidized bed. This gives granules to be applied undiluted;
   J ULV solutions (UL)
10 parts by weight of a compound according to the invention are dissolved in an organic solvent, for example xylene. This gives a product to be applied undiluted.

The active compounds can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; the intention is to ensure in each case the finest possible distribution of the active compounds according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1%.

The active compounds may also be used successfully in the ultra-low-volume process (ULV), by which it is possible to apply formulations comprising over 95% by weight of active compound, or even to apply the active compound without additives.

Various types of oils, wetters, adjuvants, herbicides, fungicides, other pesticides, or bactericides may be added to the active compounds, if appropriate not until immediately prior to use (tank mix). These agents can be admixed with the compositions according to the invention in a weight ratio of 1:10 to 10:1.

The compositions according to the invention can, in the use form as fungicides, also be present together with other active compounds, e.g. with herbicides, insecticides, growth regulators, fungicides or else with fertilizers. Mixing the compounds I or the compositions comprising them in the use form as fungicides with other fungicides results in many cases in an expansion of the fungicidal spectrum of activity being obtained.

The following list of fungicides, in conjunction with which the compounds according to the invention can be used, is intended to illustrate the possible combinations but does not limit them:
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flusilazole, hexaconazole, imazalii, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthalisopropyl,
phenylpyrroles, such as fenpiclonil or fludioxonil,
sulfur,
other fungicides, such as acibenzolar-S-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

The procedures described in the synthesis examples below were used to prepare further compounds by appropriate modification of the starting compounds. The compounds thus obtained are listed in the tables below, together with physical data.

### Example 1

### N′-[5-Chloro-6-(2,4,6-trifluorophenyl)(1,2,4-triazolo[1,5-a]pyrimidin-7-yl)]-N,N-dimethylformamidine

3 ml of dimethylformamide were initially charged in a flask and cooled to −8° C., 0.5 ml of phosphoryl chloride (POCl₃) was added dropwise and the mixture was stirred at −8° C. for 5 min. A solution of 336 mg of 7-amino-5-chloro-6-(2,4,6-trifluorophenyl)triazolo[1,5-a]pyrimidine hydrochloride in 1 ml of dimethylformamide and 0.14 ml of triethylamine was then added. After 1 h, cooling was removed and the mixture was stirred for 72 h. The reaction mixture was then poured onto ice-water and made alkaline using concentrated ammonia, and the precipitate formed was filtered off with suction. This gave, in a yield of 66%, the title compound of melting point 188-190° C.

The compounds of the formula I-A listed in Table 1 were prepared in an analogous manner (examples 2 and 3).

TABLE 1

| No. | Y—R¹ | X—R² | R³ | R⁴ | R⁵ | m.p.[° C.] |
|---|---|---|---|---|---|---|
| 1 | —N(CH₃)₂ | H | 2,4,6- | Cl | H | 188-190 |
| | | | trifluorophenyl | | | |
| 2 | 1-piperidinyl | H | 2,4,6- | Cl | H | 112-115 |
| | | | trifluorophenyl | | | |
| 3 | 1-pyrrolidinyl | H | 2,4,6- | Cl | H | 137-142* |
| | | | trifluorophenyl | | | |

m.p. melting point
*85% pure

### Example 4

### N-[5-Chloro-6-(2,4,6-trifluorophenyl)(1,2,4-triazolo[1,5-a]pyrimidin-7-yl)]acetamide

18 ml of toluene, 0.3 ml of triethylamine, 88 mg of acetyl chloride and 250 mg of 7-amino-5-chloro-6-(2,4,6-trifluorophenyl)triazolo[1,5-a]pyrimidine hydrochloride were stirred at 120° C. for 12 h. The mixture was cooled to room temperature and concentrated under reduced pressure, which gave a beige residue. This was taken up in dichloromethane, and the mixture washed with water. The organic phase was concentrated under reduced pressure, which gave, in a yield of 31%, the title compound as a beige solid of melting point 108-111° C.

### Example 5

### N-[5-chloro-6-(2,4,6-trifluorophenyl)(1,2,4-triazolo-[1,5-a]pyrimidin-7-yl)]propionamide

Using propionyl chloride instead of acetyl chloride, the process from example 4 gave the title compound of melting point 162-165° C.

Examples of the action against harmful fungi

The fungicidal action of the compounds of the formula I was demonstrated by the following experiments:

The active compounds were prepared separately as a stock solution with 0.25% by weight of active compound in acetone or DMSO. 1% by weight of the emulsifier Uniperol® EL (wetting agent having emulsifying and dispersing action based on ethoxylated alkylphenols) was added to this solution, and the mixture was diluted with water to the desired concentration.

### Use Example 1—Activity Against Early Blight Caused by Alternaria solani

Leaves of tomato plants of the cultivar “Goldene Prinzessin” were sprayed to runoff point with an aqueous suspension having the concentration of active compound stated below. The next day, the treated plants were infected with a spore suspension of Alternaria solani in a 2% strength aqueous biomalt solution having a density of 0.17×10⁶ spores/ml. The test plants were then placed in a water-vapor-saturated chamber at temperatures of from 20 to 22° C. After 5 days, the disease on the untreated, but infected plants had developed to such an extent that the infection could be determined visually.

In this test, the plants which had been treated with 250 ppm of the active compounds from example 1, 2 or 3 showed an infection of less than or equal to 1% whereas the untreated plants were 80% infected.

### Use Example 2—Activity Against Net Blotch of Barley Caused by Pyrenophora teres, 1 Day Protective Application

Leaves of potted barley seedlings of the cultivar “Igri” were sprayed to runoff point with an aqueous suspension having the concentration of active compound stated below. 24 hours after the spray coating had dried on, the plants were inoculated with an aqueous spore suspension of Pyrenophora [syn. Drechslera] teres, the net blotch pathogen. The plants were then placed in a greenhouse at temperatures between 20 and 24° C. and 95 to 100% relative atmospheric humidity. After 6 days, the extent of the mildew development was determined visually in % by the infected leaf area.

In this test, the plants which had been treated with 250 ppm of the active compounds from example 1, 2 or 3 showed an infection of ≦10%, whereas the untreated plants were 100% infected.

### Use Example 3—Activity Against Late Blight on Tomatoes Caused by Phytophthora infestans, Protective Application

Leaves of potted tomato plants were sprayed to runoff point with an aqueous suspension having the concentration of active compound stated below. The next day, the leaves were infected with an aqueous spore suspension of Phytophthora infestans. The plants were then placed in a water vapor-saturated chamber at temperatures between 18 and 20° C. After 6 days, the blight on the untreated, but infected control plants had developed to such an extent that the infection could be determined visually in %.

In this test, the plants which had been treated with 250 ppm of the active compound from example 5 showed an infection of less than or equal to 15%, whereas the untreated plants were 70% infected.

### Use Example 4—Protective Activity Against Rice Blast Caused by Pyricularia oryzae, Microtiter Test

The active compounds were formulated separately as a stock solution and with a concentration of 10 000 ppm in DMSO. The active compounds were diluted with water to the stated concentration.

50 μl of the required active compound concentration were pipetted into a mitrotiter plate (MTP). Inoculation was then carried out using 50 μl of an aqueous spore suspension of Pyricularia oryzae. The plates were placed in a water vapor-saturated chamber at temperatures of 18° C. Using an absorption photometer, the microtiter plates were measured at 405 nm on day 7 after the inoculation.

The measured parameter was compared to the growth of the active compound-free control and the blank value to determine the relative growth in % of the pathogens in the individual active compounds.

In this test, when 125 ppm of the active compound from example 4 were used, the relative growth of the spore suspension was less than or equal to 1%.

1. An azolopyrimidine compound of the formula I 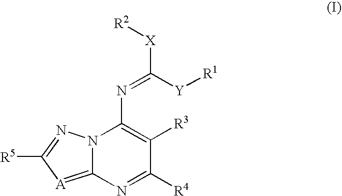
in which
A is N or C—R⁶;
X, Y independently of one another are a chemical bond or oxygen, sulfur or a group N—R⁷;
R¹, R² independently of one another are C₁-C₁₀alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl, naphthyl-C₁-C₄-alkyl, 5- or 6-membered saturated, partially unsaturated or aromatic heterocyclyl or heterocyclyl-C₁-C₄-alkyl which may in each case have 1, 2 or 3 heteroatoms selected from the group consisting of N, O and S as ring members, where some or all of the radicals mentioned as R¹, R² may be halogenated or may have 1, 2, 3 or 4 radicals R⁸, where
Y—R¹ and X—R² together with the carbon atom, to which they are attached, may also form a 5-, 6- or 7-membered saturated or unsaturated carbo- or heterocycle, where the latter may have 1, 2, 3 or 4 heteroatoms selected from the group consisting of O, S and N as ring members, where the carbo- and the heterocycle may be partially or fully halogenated or have 1, 2, 3 or 4 of the radicals R⁷ and/or R⁸; where
Y—R¹ and X—R² independently of one another may also be hydrogen, CN, NO₂ or halogen and where one of the radicals Y—R¹ and X—R² may also be OH, SH or NH₂;
R³ is C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl, a 5- or 6-membered saturated, partially unsaturated or aromatic heterocycle which may have 1, 2 or 3 heteroatoms selected from the group consisting of N, O and S as ring members,
where the radicals mentioned as R³ may be partially or fully halogenated or may have 1, 2, 3 or 4 radicals R⁹;
R⁴ is halogen, cyano, C₁-C₆-alkyl, C₁-C₆-haloalkyl, C₂-C₆-alkenyl, C₂-C₆-alkynyl, C₃-C₈-cycloalkyl, C₅-C₈-cycloalkenyl, OR¹⁰, SR¹⁰, NR¹¹R¹², CH₂NR¹¹R¹² or C(W)R¹³;
R⁵, R⁶ independently of one another are hydrogen, CN, NO₂, NH₂, CH₂NH₂, halogen, C(W)R¹³, C(═N—OR¹⁵)R¹⁴, NHC(W)R¹⁶, C₄-C₆-haloalkyl, C₁-C₄-alkyl or C₂-C₄-alkenyl;
R⁷ is hydrogen, C₁-C₆-alkyl, C₁-C₆-alkoxy, C₁-C₆-haloalkyl, C₁-C₆-haloalkoxy, C₂-C₆-alkenyl, C₂-C₆-alkenyloxy, CN or C(W)R¹⁷;
R⁸ is selected from the group consisting of halogen, cyano, nitro, OH, SH, NR¹⁸R¹⁹, C₁-C₆-alkyl, C₃-C₈-cycloalkyl, C₁-C₆-alkoxy, hydroxy-C₁-C₆-alkyl, hydroxy-C₁-C₆-alkoxy, C₁-C₆-alkoxy-C₁-C₆-alkyl, C₁-C₆-alkoxy-C₁-C₆-alkoxy, C₁-C₆-haloalkyl, C₁-C₆-haloalkoxy, C₁-C₆-alkylthio, C₂-C₆-alkenyl, C₂-C₆-alkenyloxy, C₂-C₆-alkynyl, C₂-C₆-alkynyloxy, C₁-C₆-alkylamino, C(W)R¹³, C(═N—OR¹⁵)R¹⁴, NHC(W)R¹⁶, tris-C₁-C₆-alkylsilyl and phenyl which for its part may have 1, 2 or 3 radicals selected from the group consisting of cyano, nitro, halogen, OH, C₁-C₆-alkyl, C₁-C₆-alkoxy, C₁-C₆-haloalkyl, C₁-C₆-haloalkoxy and C₁-C₆-alkylthio;
R⁹ is halogen, cyano, NH₂, NO₂, C₁-C₆-alkyl, C₃-C₈-cycloalkyl, C₁-C₆-alkoxy, C₁-C₆-haloalkyl, C₁-C₆-haloalkoxy, C₂-C₆-alkenyl, C₂-C₆-alkenyloxy, C(W)R¹³, C(═N—OR¹⁵)R¹⁴ or NHC(W)R¹⁶;
R¹⁰ is hydrogen, C₁-C₆-alkyl, C₁-C₆-haloalkyl, C₂-C₆-alkenyl or C(W)R¹³;
R¹¹, R¹² independently of one another are hydrogen, C₁-C₆-alkyl, C₂-C₆-alkenyl, C₄-C₆-alkadienyl, C₂-C₆-alkynyl, C₃-C₈-cycloalkyl, C₅-C₈-cycloalkenyl, where the radicals mentioned as R¹¹, R¹² may be partially or fully halogenated or have 1, 2, 3 or 4 radicals R⁸, where R¹¹ may also be a group C(W)R¹³ and where
R¹¹, R¹² together with the nitrogen atom, to which they are attached, may also form a 5-, 6- or 7-membered saturated or unsaturated heterocycle which may additionally have 1, 2 or 3 further heteroatoms selected from the group consisting of O, S and N as ring members, where the heterocycle may be partially or fully halogenated and/or may have 1, 2, 3 or 4 of the radicals R⁸;
R¹³ is hydrogen, OH, C₁-C₆-alkyl, C₁-C₆-alkoxy, C₁-C₆-haloalkyl, C₁-C₆-haloalkoxy, C₂-C₆-alkenyl or NR¹⁸R¹⁹;
R¹⁴, R¹⁵ independently of one another are hydrogen or C₁-C₆-alkyl;
R¹⁶, R¹⁷ independently of one another are hydrogen, C₁-C₆-alkyl, C₁-C₆-alkoxy, NH₂, C₁-C₆-alkylamino or di-C₁-C₆-alkylamino;
R¹⁸, R¹⁹ independently of one another have the meanings mentioned for R¹¹ and R¹²; and
W is oxygen or sulfur;
the tautomers of the compounds I and the agriculturally acceptable salts of the compounds I and their tautomers.

2. The compound of the formula I according to claim 1 in which at least one of the variables X or Y is a chemical bond.

3. The compound of the formula I according to claim 2 in which one of the groups Y—R¹ or X—R² is hydrogen or C₁-C₄-alkyl.

4. The compound of the formula I according to claim 1 in which both variables X and Y are a chemical bond.

5. The compound of the formula I according to claim 4 in which R¹ and R² independently of one another are selected from the group consisting of hydrogen, C₁-C₁₀-alkyl, C₁-C₁₀-haloalkyl, C₃-C₁₀-alkenyl, C₃-C₁₀-haloalkenyl, C₃-C₈-cycloalkyl, C₅-C₈-cycloalkenyl, C₃-C₈-cycloalkyl-C₁-C₁₀-alkyl, C₃-C₈-cycloalkyl-C₂-C₁₀-alkenyl, phenyl and benzyl, where the 6 lastmentioned radicals may also carry 1, 2, 3 or 4 substitutents selected from the group consisting of halogen, C₁-C₄-alkyl, C₁-C₄-haloalkyl and C₁-C₄-alkoxy.

6. The compound of the formula I according to claim 4 in which one of the groups R¹ or R² is halogen.

7. The compound of the formula I according to claim 6 in which the remaining group R¹ or R² is hydrogen, C₁-C₁₀-alkyl, C₁-C₁₀-haloalkyl, C₃-C₁₀-alkenyl, C₃-C₁₀-haloalkenyl, C₃-C₈-cycloalkyl, C₅-C₈-cycloalkenyl, C₃-C₈-cycloalkyl-C₁-C₁₀-alkyl, C₃-C₈-cycloalkyl-C₂-C₁₀-alkenyl, phenyl or benzyl, where the 6 lastmentioned radicals may also carry 1, 2, 3 or 4 substitutents selected from the group consisting of halogen, C₁-C₄-alkyl, C₁-C₄-haloalkyl and C₁-C₄-alkoxy.

8. The compound of the formula I according to claim 1 in which the group Y—R¹ is a group (NR⁷)—R¹, in which R⁷ is as defined above and R¹ is C₁-C₁₀-alkyl, C₂-C₁₀-alkenyl, C₄-C₁₀-alkadienyl, C₂-C₁₀-alkynyl, C₃-C₈-cycloalkyl, C₅-C₈-cycloalkenyl, C₅-C₁₀-bicycloalkyl, phenyl, phenyl-C₁-C₄-alkyl, naphthyl, naphthyl-C₁-C₄-alkyl and where the radicals mentioned as R¹ may be partially or fully halogenated and/or may have 1, 2, 3 or 4 radicals R⁸, or
R¹ and R⁷ together with the nitrogen atom to which they are attached form a 5- or 6-membered saturated, partially unsaturated or aromatic N-heterocycle which may have one or two further heteroatoms selected from the group consisting of O, S and N as ring member and/or may have 1, 2, 3 or 4 radicals R⁸.

9. The compound of the formula I according to claim 8 in which X is a chemical bond and R² is hydrogen or C₁-C₄-alkyl.

10. The compound of the formula I according to claim 8 in which the group (NR⁷)R¹ is C₁-C₆-alkylamino, di-C₁-C₆-alkylamino or a 5- or 6-membered saturated heterocyclyl which is attached via nitrogen, which optionally has a further heteroatom selected from the group consisting of N, O and S as ring atom and which optionally carries, 1, 2, 3 or 4 substitutents R⁸ selected from the group consisting of halogen and C₁-C₄-alkyl.

11. The compound of the formula I according to claim 1 in which R³ is a phenyl ring which has 1, 2, 3 or 4 radicals R⁹.

12. The compound of the formula I according to claim 11 in which R³ is a group of the formula 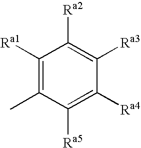
in which
R^{a1} is fluorine, chlorine, trifluoromethyl or methyl;
R^{a2} is hydrogen, chlorine or fluorine;
R^{a3} is hydrogen, CN, NO₂, fluorine, chlorine, C₁-C₄-alkyl, C₁-C₄-alkoxy or a group C(W)R^{13a} in which R^{13a} is C₁-C₄-alkoxy, NH₂, C₁-C₄-alkylamino or di-C₁-C₄-alkylamino;
R^{a4} is hydrogen, chlorine or fluorine;
R^{a5} is hydrogen, fluorine, chlorine or C₁-C₄-alkyl.

13. The compound of the formula I according to claim 1 in which R⁴ is halogen, CN, methyl or methoxy.

14. The compound of the formula I according to claim 13 in which R⁴ is halogen.

15. The compound of the formula I according to claim 1 in which R⁵ is hydrogen.

16. The compound of the formula I according to claim 1 in which A is N.

17. The compound according to claim 1 in the form of the tautomers of the formula II 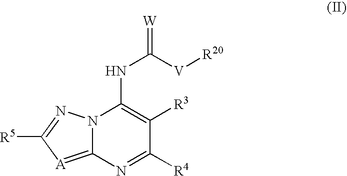
in which A, R³, R⁴ and R⁵ have the meanings given above for formula I,
V is a chemical bond or is oxygen, sulfur or a group N—R⁷;
W^{a} is O, S or a group N—R²¹;
R²⁰ has one of the meanings given in formula I for R¹ or R²;
R²¹ has one of the meanings given in formula I for R¹ or R² or is hydrogen; and
if W^{a} is N—R²¹, V—R²⁰ and N—R²¹ together with the carbon atom, to which they are attached, may form a 5-, 6- or 7-membered unsaturated heterocycle, where the latter may have 1, 2, 3 or 4 heteroatoms selected from the group consisting of O, S and N as ring members, may be partially or fully halogenated or have 1, 2, 3 or 4 of the radicals R⁸ mentioned above.

18. The use of a compound of the formula I according to claim 1 or an agriculturally acceptable salt thereof for controlling phytopathogenic fungi.

19. A composition for controlling phytopathogenic fungi, which composition comprises at least one compound of the formula I according to claim 1 and/or an agriculturally acceptable salt of I and at least one liquid or solid carrier.

20. A method for controlling phytopathogenic fungi, which method comprises treating the fungi or the materials, plants, the soil or seeds to be protected against fungal attack with an effective amount of a compound of the formula I according to claim 1 and/or with an agriculturally acceptable salt of I.
